(19) United States
(12) Patent Application Publication
(10) Pub. No.: US 2010/0317846 A1
(43) Pub. Date: Dec. 16, 2010

(54) Production Of L-Iduronate Containing Polysaccharides

(75) Inventors: Steen Uldall Hansen, Manchester (GB); Gordon Jayson, Manchester (GB); John Gardiner, Manchester (GB); Marek Barath, Manchester (GB)

Correspondence Address: LERNER, DAVID, LITTENBERG,;KRUMHOLZ & MENTLIK, 600 SOUTH AVENUE WEST, WESTFIELD, NJ, 07090, US

(73) Assignee: THE UNIVERSITY OF MANCHESTER, Manchester (GB)

(21) Appl. No.: 12/866,091

(22) PCT Filed: Feb. 4, 2009
(86) PCT No.: PCT/GB09/00300

§ 371 Date: Aug. 4, 2010

(30) Foreign Application Priority Data
Feb. 4, 2008 (GB) 0801977.0

(51) Int. Cl. C07H 1/00; C07H 3/04; C08B 37/00; C07H 3/02

(52) U.S. Cl. 536/123.13; 536/123.1; 536/124; 536/1.11

(57) ABSTRACT

A process for the production of polysaccharide (20) from disaccharide (10) and saccharide (21), wherein R¹ to R¹⁰ are each independently the same or different protecting groups; R is an optionally substituted aryl group or an optionally substituted saturated or unsaturated alkyl group; X is selected from the group consisting of hydrogen, alkyl and amino; Y is selected from the group consisting of a protecting group and one or more saccharide residues; and n is a positive integer; and further wherein said process comprises removal of the R⁷ protecting group and reaction of the deprotected C4-oxygen atom of compound (21) with the C1-carbon atom of the 1-ido moiety of compound (10).

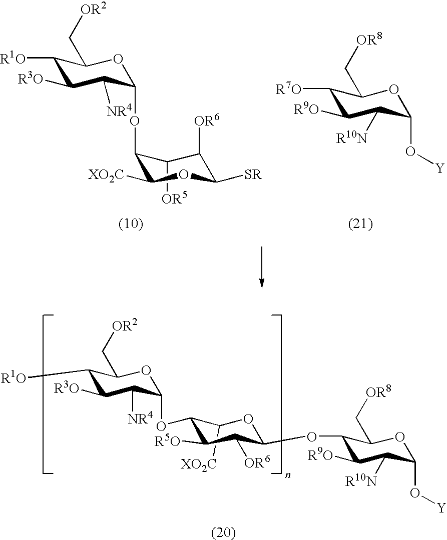

The present invention relates to the production of polysaccharides containing the 1-iduronate subunit, particularly but not exclusively to heparin-type polysaccharides, and also to new intermediate compounds and processes developed during the production of said polysaccharides.

Carbohydrates represent one of the major classes of biomolecules and are critical to the regulation of a large number of biological processes and pathways. A common monosaccharide unit found in many carbohydrates is the 1-Iduronate residue A (where, for example, R═SO₃⁻) which is related to 1-Iduronic acid B.

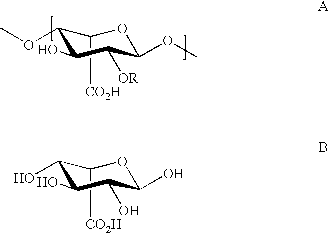

1-Iduronic acid is a ‘rare’ sugar in that it possesses the 1-configuration at the C5 position whereas all other common, readily available sugars possess the opposite D-configuration at C5. As a result, commercially viable syntheses of 1-Iduronic acid, its derivatives and polysaccharides containing 1-Iduronate subunits from readily available sugars have not yet been developed. Synthesis of 1-Iduronic acid and its derivatives is therefore of significant commercial importance since these compounds are not available at viable cost from natural sources.

Examples of biologically important 1-Iduronate-containing polysaccharides are heparan sulfate and heparin, which play a central role in many different biological processes including anti-coagulation, angiogenesis, cell growth and migration.

Heparan sulfate and heparin exist as complex heterogeneous mixtures of polysaccharide chains of varying length. The chains are principally composed of repeating disaccharide units as shown below.

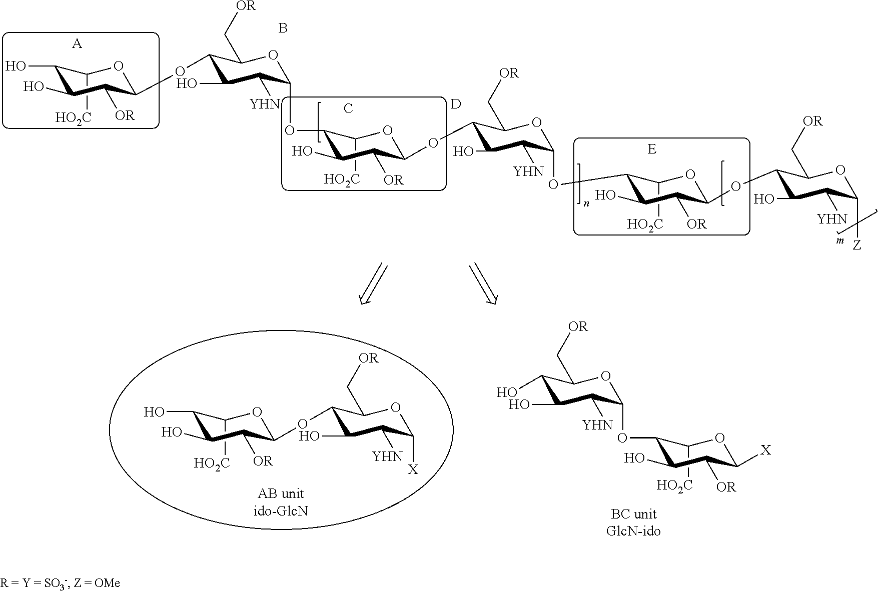

The repeating unit may be regarded as either an ‘AB’ glycosaminoglycan unit in which adjacent sugar rings are linked via a α(1→4)-glycosidic bond or a ‘BC’ glycosaminoglycan unit containing an α(1→4)-glycosidic bond. In each case, the disaccharide unit contains an 1-iduronate moiety.

Medicinal drugs that promote or inhibit the function of heparan sulfate/heparin by mimicking or competitively inhibiting the function of heparan sulfate/heparin could potentially be used in a number of diseases that affect the general population. The therapeutic potential of these compounds includes cardiology/vascular medicine (anti-coagulation), cancer (angiogenesis and tumour growth), diabetic retinopathy (angiogenesis) and rheumatoid arthritis (angiogenesis in the pannus). By way of example, heparan sulfate and heparin are known to be involved in the regulation of the fibroblast growth factor FGF-2. In view of the fact that FGF-2 has been implicated in angiogensis, FGF-2 inhibitory heparin systems have great potential as anti-angiogenic/anti-tumour agents. Ovarian cancer is the commonest cause of gynaecological cancer death and accounts for 5000 lives a year in the UK. Although surgery and chemotherapy improve survival¹⁻³, improvements are needed both in remission induction and in maintenance therapy.

A large amount of data are accumulating which suggest that FGFs are an important target in ovarian cancer. They enhance tumour growth and tumour angiogenesis and, significantly, they are strongly implicated in resistance to VEGF inhibitors⁴⁻¹³, an area of increasing clinical importance. The FGFs therefore fulfil the criteria for an important target in ovarian cancer.

The FGF signalling system comprises 22 growth factors and 4 signal transducing receptors. The extracellular domains of the receptors consist of 3 immunoglobulin folds that can be differentially spliced to produce several receptor isoforms. This has functional significance for FGF7 as only the FGFR2IIIb isoform binds the cytokine¹⁴ and for FGF2 that acts principally on the IIIc isoforms. From the biological perspective this is important as published data have shown that the transformation of prostatic epithelium to adenocarcinoma is associated with a loss of expression of this receptor^{15,16}. In a comprehensive study of ovarian cancer⁸ a receptor isotype switch from FGFR2IIIc to FGFR2IIIb in malignant epithelial ovarian cancer has been demonstrated. In unpublished studies it has now been shown that the ligands for this receptor, FGF 3 and 7 are mitogenic cytokines that endow the ES2 and A2780 ovarian cancer cell lines with resistance to cisplatin. Immunohistochemical data confirmed the near universal expression of these cytokines in ovarian cancer tissue. Since it is known from published studies that FGF2 is a relevant target in the ovarian cancer endothelium⁴, it is therefore appropriate to develop inhibitors of FGF2, 3 and 7 for the treatment of ovarian cancer.

FGFS. 2^{17,18}, 3¹⁹ and 7^{20,21} are dependent on HS for their biological activity and in previous studies it has been shown that heparin octasaccharides have the capacity to inhibit FGF2 in vitro²² and FGF2-induced angiogenesis in vivo²³. Taken together this implies that FGFs 2, 3 and 7 are implicated in ovarian cancer angiogenesis and growth. These growth factors and their heparn sulfate co-receptor are therefore appropriate targets for treatment. More recently, pathological studies in Alzheimer's disease have suggested that heparan sulfate may be pathophysiologically relevant and there is therefore a further potential market for saccharide based drugs in that setting.

Commercial production of drugs that promote or inhibit the function of heparan sulfate and/or heparin will therefore be reliant on the development of viable syntheses of appropriate saccharide building blocks and their derivatives.

Sulfation at several sites in the saccharide backbone is normally clustered in domains of anionic charge that engage proteins through ionic forces, principally through lysine and arginine residues. The glycosaminoglycan chain attains flexibility through the presence of iduronate residues which can adopt a number of conformations, facilitating ligand binding²⁴. The biosynthesis of HS occurs as a post-translational modification of cell surface and extracellular matrix proteins in the Golgi apparatus, generating HS chains that commonly bear sulfated domains composed of sugar sequences modified at the N- and 6-O-positions in glucosamine and the 2-O-position in iduronate. The enzymes that bring about these modifications are present in ovarian cancer tissue especially within the tumour vasculature.

FGF2 binds a sequence of HS that contains both N- and 2-O sulfation²⁵. However, the HS-FGF interaction alone is insufficient for biological activity and at least one 6-O-sulfate group is needed for the interaction between FGF-2 and its receptor^{18,26-29}, suggesting that HS might be involved in the formation of a tri-molecular signalling complex. This is mediated through the capacity of HS to bind both the cytokine and its receptor; the HS binding-site in the receptor residing in an 18 amino acid sequence lying between Ig domains 2 and 3³⁰. Two x-ray crystallographic studies have implicated this sequence in the formation of a tri-molecular complex comprising heparan sulfate, FGF and the signalling receptor^{31,32} although the exact stoichiometry of this complex is not firmly established³³ and other parts of the receptor appear to influence the affinity for HS³⁴.

A pentasaccharide sequence had been identified as the minimum binding sequence for FGF-2 (-hexuronic acid-glucosamine N-sulfate-hexuronic acid-glucosamine N-sulfate-iduronic acid 2-O-sulfate-)³⁵. However, further compositional analysis suggested that the affinity of saccharide species increases as the proportion of iduronate increases, presumably due to the increased flexibility conferred on the HS chain by iduronate³⁶, thus permitting a conformational change in the HS sequence upon ligand binding²⁴. In general most studies have shown that activation of FGF2 requires species that contain at least 10 saccharide residues²² while others have observed some cytokine activation with shorter sequences³⁷ and in part these differences may be accounted for by the heterogeneity of saccharide preparation and the different models under investigation³⁷. Importantly, recent binding studies' and data emerging from pre-clinical and clinical studies³⁸⁻⁴⁰ of the highly charged saccharide, PI-88 (sulfo-manno-pentaose) suggest that charge density is critical to the inhibitory potential of oligosaccharides. Taken together these data suggest that Heparin oligosaccharides offer the potential to act as inhibitors of FGFs 2, 3 and 7 signal transduction for the treatment of ovarian cancer. It would be highly desirable to be able to impose an artificially high charge density on the heparin backbone that is likely to disrupt ligand-receptor interactions more efficiently than a similar charge density on a non-physiological mannan template.

New methodology for the synthesis of 1-iduronic acid derivatives was developed via novel stereoselective synthesis of the intermediate cyanohydrin 3′ prepared from D-glucose 1′ via the x-D-xylo-dialdose 2′ as shown below. This is the subject of the applicant's published co-pending International patent application (WO2006/129075).

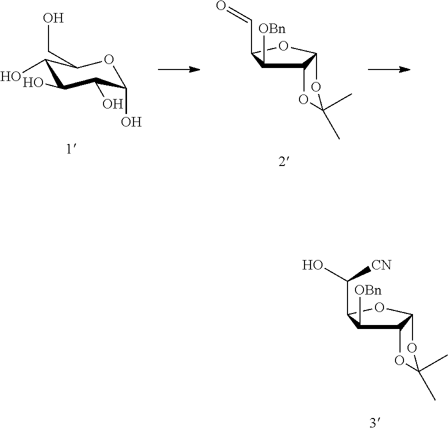

The above conversion of compound 2′ to compound 3′ is effected by reacting compound 2 with cyanide ions in the presence of magnesium ions.

The methodology elucidated in WO2006/129075 marked a significant advance in the synthesis of saccharide polymers containing the iduronate subunit, however, it would be desirable to undertake further optimization of the ido unit to ensure that the most efficient donor and acceptor derivatives/anomeric groups were introduced, with regards to both coupling efficiencies in oligosaccharide synthesis and high stereocontrol in those coupling reactions.

An object of the present invention is to develop improved methods for use in the production of iduronate-containing polysaccharides and related compounds.

According to a first aspect of the present invention there is provided a process for the production of polysaccharide 20 from disaccharide 10 and saccharide 21

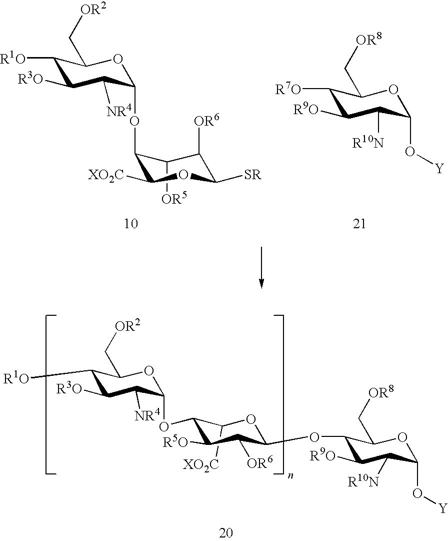

wherein R¹ to R¹⁰ are each independently the same or different protecting groups; R is an optionally substituted aryl group or an optionally substituted saturated or unsaturated alkyl group; X is selected from the group consisting of hydrogen, alkyl and amino; Y is selected from the group consisting of a protecting group and one or more saccharide residues; and n is a positive integer; and
further wherein said process comprises removal of the R⁷ protecting group and reaction of the deprotected C4-oxygen atom of compound 21 with the C1-carbon atom of the 1-ido moiety of compound 10, that is, the C1-carbon atom bonded to the SR group.

A significant, yet surprising, advantage associated with thioglycoside 10 is that it facilitates highly efficient coupling with appropriate donor compounds having the generic formula 21 so as to provide polysaccharides of generic formula 20. The thioglycoside 10 is a more stable compound than analogous glycoside compounds in which the anomeric group is other common leaving groups, such as a trichloroacetimidate (TCA) group. The thioglycoside 10 can be prepared, using methodology described below, on a large scale and can be safely stored over extended periods of time for use when desired.

R is an optionally substituted aryl group or an optionally substituted saturated or unsaturated alkyl group. It is preferred that the group R contains 1 to 12 carbon atoms, more preferably 2 to 10 carbon atoms, and most preferably 4 to 8 carbon atoms. In a preferred embodiment, R is a substituted or unsubstituted C₄ to C₁₀ aryl group, such as an optionally substituted phenyl group. R may be an optionally substituted linear or branched alkyl group, such as a C₁ to C₈ alkyl group, e.g. a methyl, ethyl or propyl group.

The group R may be unsubstituted or alternatively may contain any desirable degree of substitution, that is the R group may contain one or more substituent (i.e. non-hydrogen) groups on one or more carbon atoms contained in group R. Preferred substituent groups include, but are not limited to, alkyl, alkoxy and nitro groups.

Where R is an aryl group, a preferred substitution pattern is to provide an alkyl, alkoxy or nitro group at the para-position relative to the carbon atom via which group R is bonded to the sulfur atom of the —C1-SR group of the 1-ido moiety. It is particularly preferred that the —C1-SR group is a —C1-SPh group, that is, R is preferably a phenyl group, which may be further substituted with one or more substituents (non-hydrogen atoms), including a para-nitro (—NO₂) group, a para-methoxy (—OMe) group or a para-methyl (-Me) group.

In a further preferred embodiment of the present invention groups R and X may together define a linking group, RX, in which the —C1-S— group of the 1-ido moiety is linked, via group RX, to one of the C6-oxygen atoms of the same 1-ido moiety, such that the C1-sulfur atom is linked to one of the carboxyl group oxygen atoms prior to the coupling reaction taking place.

It is preferred that R⁶ is a benzoyl group. It has been unexpectedly determined that employing a benzoyl protecting group on the 2-O atom of the thioglycoside 10 enhances the desired stereoselectivity of the coupling reaction with donor compound 21. At least one, preferably both, of R¹ and R⁷ may be a para-methoxybenzyl (PMB) protecting group. Preferably at least one of R², R³, R⁵, R⁸ and R⁹ is an aryl group, such as a benzyl group. More preferably most, and still more preferably all, of R², R³, R⁵, R⁸ and R⁹ is a benzyl group.

Any desirable protecting group may be provided on each of the nitrogen atoms of thioglycoside 10. Preferably at least one of NR⁴ and NR¹⁰ is an azide group, such as N₃. More preferably both of NR⁴ and NR¹⁰ is an N₃ group.

The process according to the first aspect of the present invention comprises removal of the R⁷ protecting group to facilitate reaction, i.e. coupling, of disaccharide 10 with saccharide 21. Any appropriate agent may be used to remove the R⁷ protecting group, depending upon the chemical nature of the R⁷ protecting group and the nature of the other groups present in saccharide 21. In a preferred example, the protecting group R⁷ may be removed by using ceric ammonium nitrate (CAN) or 2,3-Dichloro-5,6-Dicyanobenzoquinone (DDQ) at around room temperature (r.t.). This agent is particularly preferred when R⁷ is a PMB protecting group.

Reaction of disaccharide 10 with saccharide 21 may be effected in the presence of a coupling promotor. It will be appreciated that any appropriate coupling promotor can be employed, depending upon various factors, such as the nature of the chemical groups present in the sugars 10, 21 being coupled. A preferred coupling promotor is N-iodosuccinimide (NIS) and silver trifluoromethane sulfonate (AgOTf). The coupling reaction of disaccharide 10 with saccharide 21 may be effected at any suitable temperature, and it has been determined that temperatures below around room temperature (r.t.), e.g. around 0° C., may be particularly suitable. It is particularly preferred that the coupling reaction is effected at a temperature of around 0° C. when the coupling promotor is NIS/AgOTf.

In order to facilitate the production of longer polysaccharides, i.e. polysaccharides of formula 20 in which n is two or more, once a polysaccharide of formula 20, in which n is one, has been produced by coupling a first disaccharide unit of formula 10 with a first saccharide unit of formula 21, polysaccharide 20 can then be deprotected by removal of the R¹ protecting group and thereby activated for reaction with at least one further unit of disaccharide 10 to provide a polysaccharide of formula 20 in which n is two or more, that is, n is increased by one for the or each further disaccharide unit 10 that is coupled to polysaccharide 20. In a preferred embodiment of the process of the present invention the process further comprises one or more polysaccharide elongation steps, each step comprising removal of the R¹ protecting group of polysaccharide 20 and reaction of the deprotected C4-oxygen atom of polysaccharide 20 with the C1-carbon atom of the 1-ido moiety of a further unit of compound 10, n being increased by one for the or each polysaccharide elongation step. Thus, n represents the total number of units of disaccharide 10 added to saccharide unit 21, n may therefore take any appropriate positive integer value, such as but not limited to, 1, 2, 3, 4 or more depending upon the number of units of disaccharide 10 added.

The applicant's published co-pending International patent application (WO2006/129075) provided an entry to octasaccharide assembly in a ‘geometric’ synthetic manner, but this methodology relies on divergence and manipulation of tetrasaccharides (thus quite late in synthesis). The process according to the first aspect of the present invention reiterates use of disaccharide unit 10 only, which provides higher overall coupling efficiencies by not relying on reiterative use of longer sequences. This has been strongly vindicated in that the iterative disaccharide assembly approach has proven to have better coupling efficiencies throughout (at least in part due to the use of a benzoyl group at the R⁶ position of thioglycoside 10) and importantly establishes a capped glycoside end at the reducing terminus, thereby avoiding the need to convert a labile terminal group towards the end of the coupling process, which is required in the applicant's earlier methodology presented in WO2006/129075 which may not be as efficient as one might require.

In part to circumvent analytical difficulties from anomerically-mixed reducing terminal units and to develop a more materially efficient route, disaccharide iterative chemistry was developed by first coupling thioglycoside 10 to the reducing terminal glucoside 21. This proved an important advantage since the glucoazide glycoside polysaccharide 20 is obtained as a single (alpha) anomer ensuring more robust analysis during oligosaccharide assembly.

Realization of the planned iterative process for disaccharide attachment proceeded with high yields and stereoselectivity, converting initial trisaccharide 20 in which n is one and Y is a protecting or capping end group, into higher polysaccharides of general formula 20 in which n is two and then subsequently three, with 80% yields at each of the two intermediate glycosidic coupling steps. By way of example, this methodology allowed synthesis of 1-200 mg amounts of a heptasaccharide of general formula 20. The development of a short linear iteration with good efficiencies makes large-scale synthesis now viable and the overall scalability relies on stocks of a single disaccharide unit, and it is this which makes the overall route a considerable advance for scalability objectives.

A further aspect of the present invention provides a polysaccharide compound of formula 20′

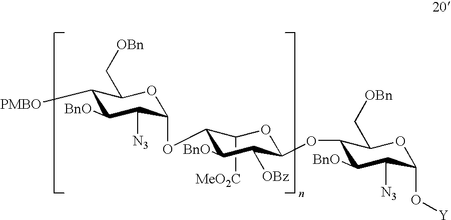

wherein Y is a protecting group or one or more saccharide residue; and n is a positive integer.

The terminal chemical group, Y, may be any desirable chemical protecting group, and may be considered as a capping end group, preventing further coupling at the terminal end of polysaccharide 20. In a first preferred embodiment of the first aspect of the present invention, generic compound 21 incorporates a Y group, R¹⁴, that is a substituted or unsubstituted aryl group or a substituted or unsubstituted saturated or unsaturated alkyl group (preferably R¹⁴ is a lower alkyl group, e.g. a C₁-C₆ alkyl group, such as a methyl group) thereby providing a preferred compound 12,

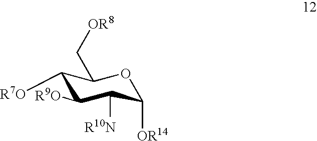

which, following removal of the R⁷ protecting group, can be coupled to compound 10 to provide a first preferred polysaccharide 15 (which represents a first preferred embodiment of polysaccharide 20 defined above), as shown below.

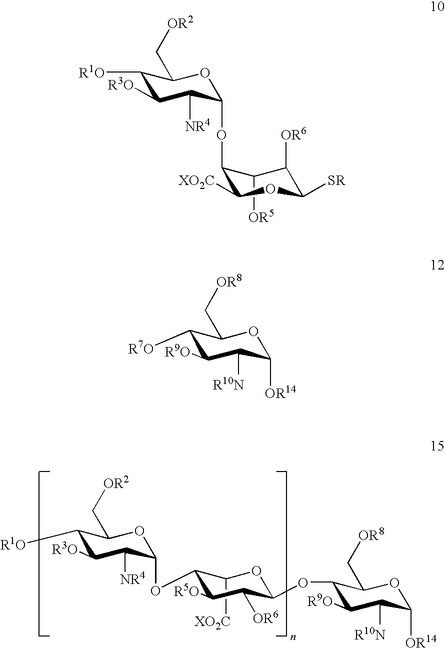

The above coupling process provides a polysaccharide 15, which represents a further aspect of the present invention,

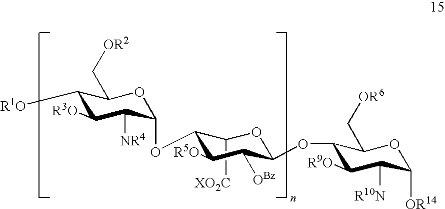

wherein R¹ to R⁵, R⁸ to R¹⁰ are each independently the same or different protecting groups; R¹⁴ is a substituted or unsubstituted aryl group or a substituted or unsubstituted saturated or unsaturated alkyl group; X is selected from the group consisting of hydrogen, alkyl and amino; and n is a positive integer.

Deprotection of monosaccharide 12, which is required before it can be coupled to compound 10, may be achieved in any convenient way depending upon the nature of the R⁷ protecting group. This process provides an activated monosaccharide 12A in which the previously protected and unreactive R⁷⁰— group is replaced with an HO— group which can react with the C1-carbon atom of the 1-ido moiety of compound 10.

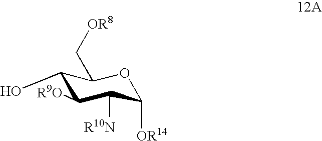

Throughout the present application, specific embodiments of compounds falling within a particular general formula will take the same compound number as the general formula but suffixed with a prime (′). Thus, in the process set out below, compounds 10′, 12′ and 15′ represent specific preferred embodiments of compounds of general formula 10, 12 and 15 respectively as set out above. In a further aspect of the present invention there is provided a process for the production of preferred polysaccharide 15′ from disaccharide 10′ and monosaccharide 12′

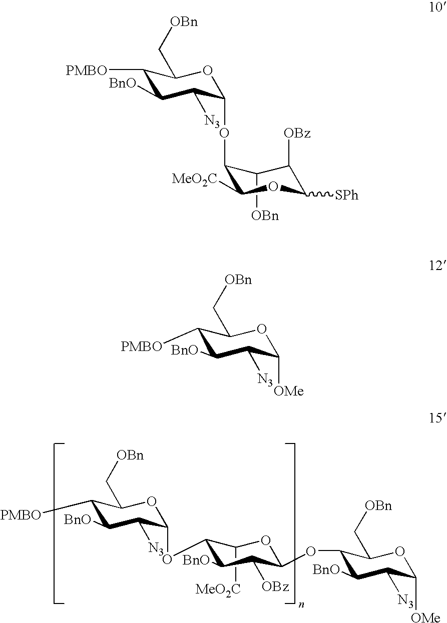

wherein n is a positive integer; and
further wherein said process comprises removal of the PMB protecting group of compound 12′ and reaction of the deprotected C4-oxygen atom of compound 12′ with the C1-carbon atom of the 1-ido moiety of compound 10′.

In the above process where the specific monosaccharide 12′ is employed, deprotection of compound 12′ involves removal of a PMB protecting group, which may be achieved, for example, by using ceric ammonium nitrate (CAN) or PDQ optionally, at around room temperature (r.t.). This process provides an activated monosaccharide 12A′ in which the previously protected O4 bearing a PMB is deprotected to provide the 4-OH group which can react with the C1-carbon atom of the 1-ido moiety of compound 10′.

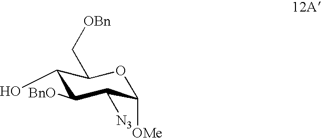

Coupling of compounds 10′ and 12′ provides a specific polysaccharide 15′, which represents another aspect of the present invention,

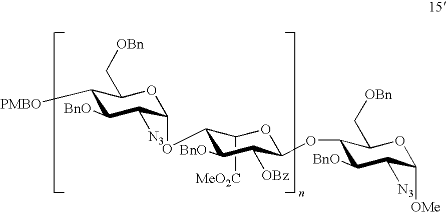

wherein n is a positive integer.

The polysaccharide 15 (e.g. 15′) produced as described above by coupling one unit of compound 10 to one unit of compound 12, e.g. 12′ (or more specifically, deprotected compound 12A, e.g. 12A′) may be reacted with one or more further units of disaccharide 10 (e.g. 10′) to provide a polysaccharide 15 (e.g. 15′) wherein n is increased by one for the or each further disaccharide unit 10 (e.g. 10′). Polysaccharide 15′ must be deprotected before the polysaccharide chain can be elongated. Thus, it is preferred that the process further comprises one or more polysaccharide elongation steps, each step comprising removal of the R¹ (e.g. PMB) protecting group of polysaccharide 15 (e.g. 15′) and reaction of the deprotected C4-oxygen atom of polysaccharide 15 (e.g. 15′) with the C1-carbon atom of the 1-ido moiety of a further unit of compound 10 (e.g. 10′), n being increased by one for the or each polysaccharide elongation step.

In an alternative preferred embodiment of the first aspect of the present invention Y is a monosaccharide unit 22 as shown below,

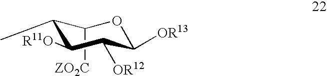

wherein at least one of R¹¹, R¹² and R¹³ is a protecting group; and Z is selected from the group consisting of hydrogen, alkyl and amino. Monosaccharide unit 22 is connected to saccharide 21 via the C4-carbon atom of unit 22 (shown above with a single chemical bond extending therefrom with no terminal chemical group).

R¹² may be any desirable protecting group, and a particularly preferred protecting group is a benzoyl group. At least one, preferably both, of R¹¹ and R¹³ is an alkyl or aryl group. R¹¹ may be a benzyl group and R¹³ may be an alkyl group, preferably a lower alkyl group, such as a methyl group. The carboxylate group (—COOZ) may be an ester functional group, wherein Z is an alkyl group, such as a methyl group.

In a second preferred embodiment of the first aspect of the present invention, generic compound 21 incorporates a Y group that has the structure of monosaccharide unit 22 defined above, thereby providing a second preferred compound 14,

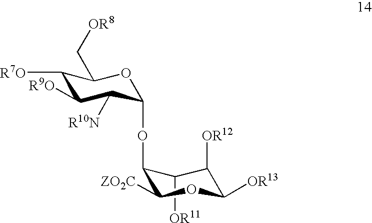

which, following removal of the R⁷ protecting group, can be coupled to compound 10 to provide a preferred polysaccharide 16 (which represents a second preferred embodiment of polysaccharide 20 defined above), as shown below.

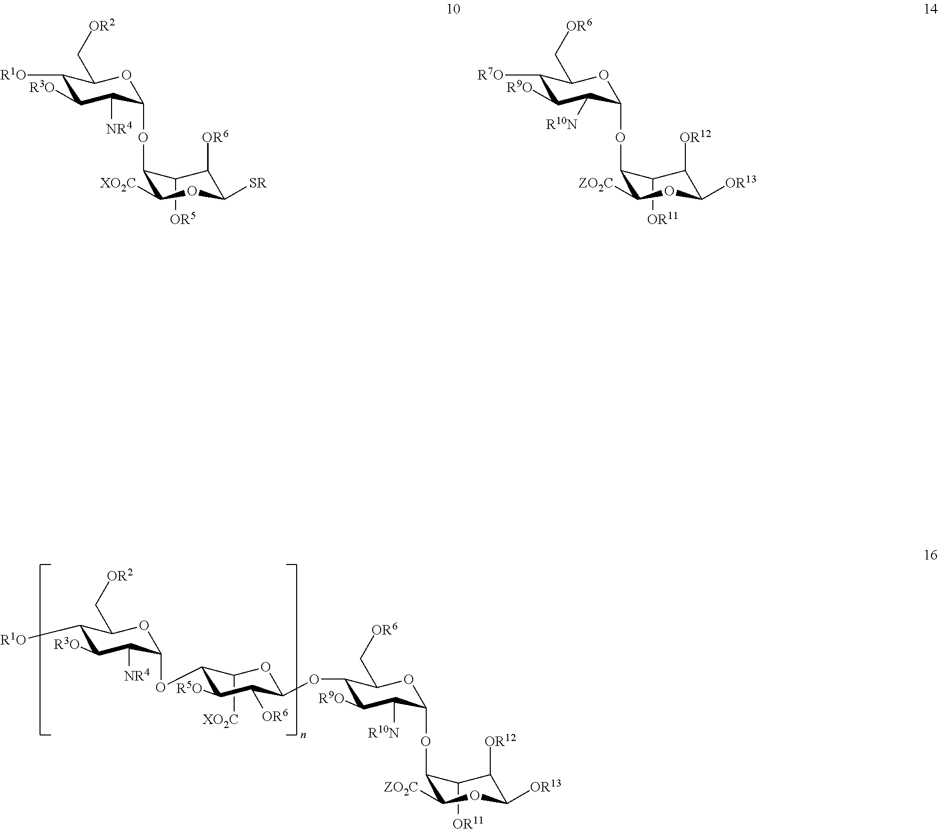

Deprotection of disaccharide 14, which is required before it can be coupled to compound 10, may be achieved in any convenient way depending upon the nature of the R⁷ protecting group. This process provides an activated disaccharide 14A in which the previously protected and unreactive R⁷⁰— group is replaced by an HO— group which can react with the C1-carbon atom of the 1-ido moiety of compound 10.

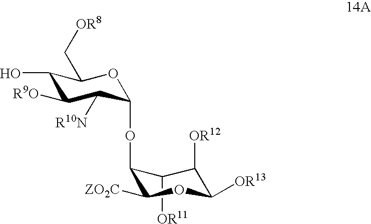

The above coupling process provides a polysaccharide 16, which represents a further aspect of the present invention,

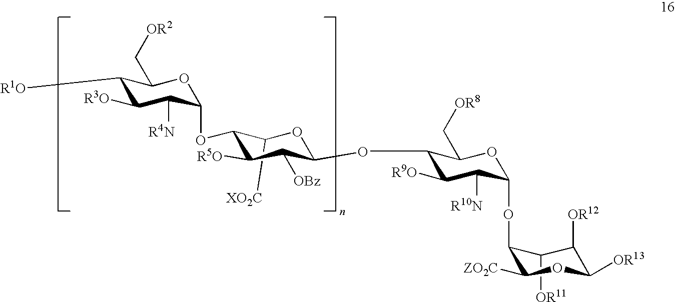

wherein each of R¹ to R⁵, R⁸ to R¹³ are each independently the same or different protecting groups; X and Z are each independently selected from the group consisting of hydrogen, alkyl and amino; and n is a positive integer.

In a further aspect of the first aspect of the present invention there is provided a process for the production of polysaccharide 16′ from disaccharide 10′ and disaccharide 14′

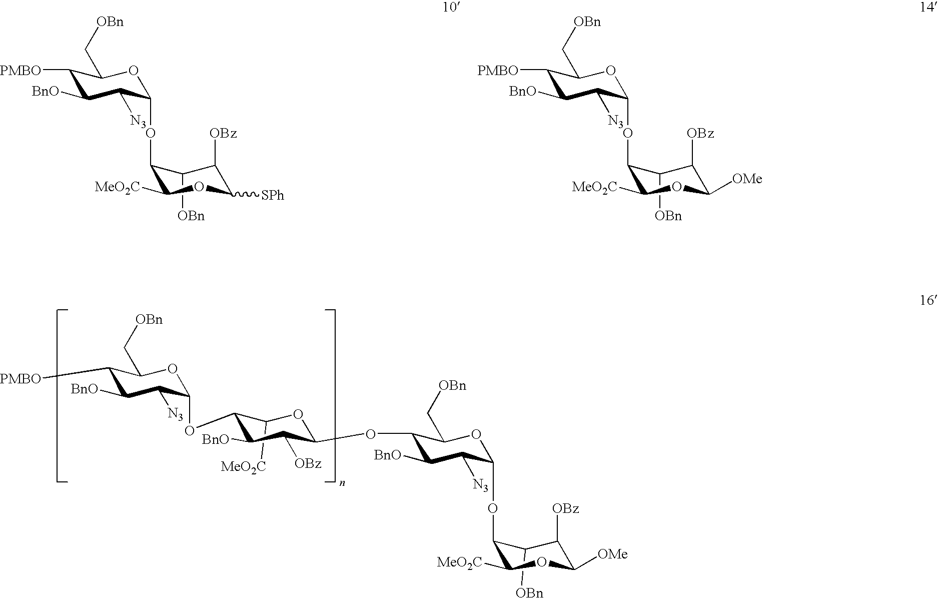

wherein n is a positive integer; and
further wherein said process comprises removal of the PMB protecting group of compound 14′ and reaction of the deprotected C4-oxygen atom of the compound 14′ with the C1-carbon atom of the 1-ido moiety of compound 10′.

In the above process where the specific monosaccharide 14′ is employed, deprotection of compound 14′ involves removal of a PMB protecting group, which may be achieved, for example, by using ceric ammonium nitrate (CAN) optionally at around room temperature (r.t.). This process provides an activated monosaccharide 14A′ in which the previously protected O4 bearing a PMB is deprotected to provide the 4-OH group which can react with the C1-carbon atom of the 1-ido moiety of compound 10′.

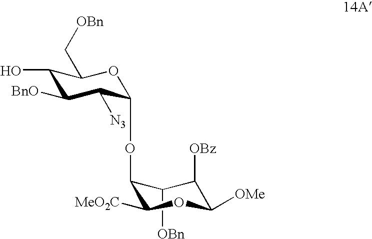

Coupling of compounds 10′ and 14A′ provides a specific polysaccharide 16′, which represents another aspect of the present invention,

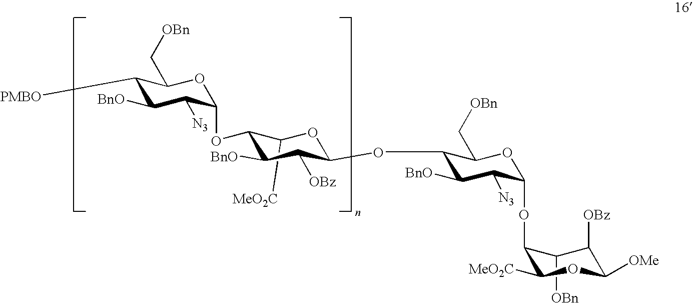

wherein n is a positive integer.

Polysaccharide 16 (e.g. 16′) may be reacted with one or more further units of disaccharide 10 (e.g. 10′) to provide a polysaccharide 16 (e.g. 16′) wherein n is increased by one for the or each further disaccharide unit 10 (e.g. 10′). It is preferred that the process according to the first aspect of the present invention further comprises one or more polysaccharide elongation steps, each step comprising removal of the R¹ (e.g. PMB) protecting group of polysaccharide 16 (e.g. 16′) and reaction of the deprotected C4-oxygen atom of polysaccharide 16 (e.g. 16′) with the C1-carbon atom of the 1-ido moiety of a further unit of compound 10 (e.g. 10′), n being increased by one for the or each polysaccharide elongation step.

In another aspect, the present invention provides a process for the production of disaccharide 10 by the reaction of compound 8 with compound 9

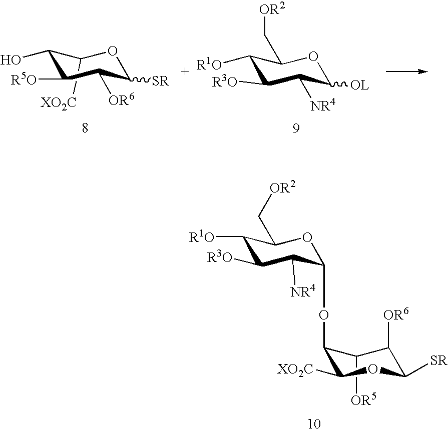

wherein R is an optionally substituted aryl group or an optionally substituted saturated or unsaturated alkyl group; R¹ to R⁶ are each independently the same or different protecting groups; X is selected from the group consisting of hydrogen, alkyl and amino; and L is a leaving group.

Core unit, 10, contains a stable donor terminus (—SR), which may for example be a thiophenyl group (—SPh). Coupling of glucoazide donor 9 and ido acceptor 8 proceeds stereospecifically and in high yield and affords multigram amounts of disaccharide 10.

Preferably R⁶ is a benzoyl group. R⁵ may be an alkyl group or an aryl group, preferably R⁵ is a benzyl group.

The leaving group, L, may be any appropriate leaving group. A particularly preferred leaving group, L, is a trichloroacetimidate group.

While R¹ may be any suitable protecting group, it is preferred that it is a PMB group. It is preferred that R¹ is a PMB group since this represents a readily removable 4-0 terminus protecting group, which can be exploited in subsequent coupling reactions, such as the process set out above in respect of the first aspect of the present invention. Moreover, one, or preferably both of R² and R³ is an alkyl or aryl group, such as a benzyl group.

Reaction of compound 8 with compound 9 is preferably effected in the presence of a coupling promotor, such as trimethylsilyl trifluoromethane sulfonate (TMSOTf). The reaction may be effected at any appropriate temperature, and is preferably effected at a temperature that is below around room temperature (r.t.). A particularly suitable reaction temperature when using TMSOTf as coupling promotor is around −20 to −30° C.

In a preferred embodiment the process according to the current aspect of the present invention comprises reacting compound 8′ with compound 9′ to provide disaccharide 10′

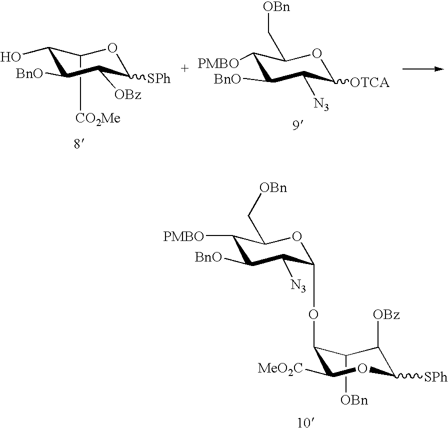

A related aspect of the present invention provides disaccharide 10

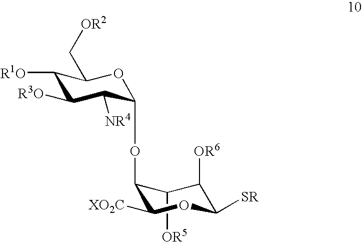

wherein R is an optionally substituted aryl group or an optionally substituted saturated or unsaturated alkyl group; R¹ to R⁶ are each independently the same or different protecting groups; and X is selected from the group consisting of hydrogen, alkyl and amino. Preferably R is an aryl group, such as a phenyl group, and R⁶ is a benzoyl group.

Another related aspect provides disaccharide 10′

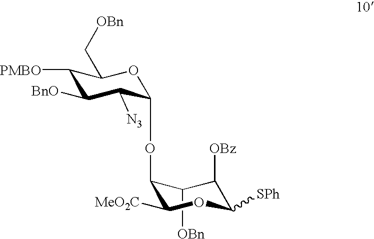

A means for providing compound 8 employed above from compound 7, as shown below, represents a further aspect of the present invention

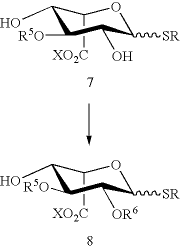

wherein R is an optionally substituted aryl group or an optionally substituted saturated or unsaturated alkyl group; R⁵ and R⁶ are each independently the same or different protecting groups; X is selected from the group consisting of hydrogen, alkyl and amino; and said process comprises adding protecting group R⁶ to the 2-O atom of compound 7.

Preferably R is an aryl group, such as a phenyl group.

Preferably R⁶ is a benzoyl group.

Addition of said protecting group may be effected by reacting compound 7 with a benzoyl halide compound, e.g. BzCl, in the presence of a promotor species.

Any appropriate promotor may be used, and it is preferred that said promotor species is dibutyl tin oxide.

Conveniently, compound 7 is exposed to said promotor species at around room temperature (r.t.) or higher, e.g. up to around 50° C. prior to being reacted with the benzoyl halide. This process can be conducted at any appropriate temperature. While the reaction can be effected at around 0° C., particularly suitable temperatures have generally been determined to be above around room temperature (r.t) or higher, preferably around 50° C.

In a related aspect of the present invention there is provided a process for the production of compound 8′ from compound 7′ by adding a benzoyl group to the 2-O atom of compound 7′ as shown below.

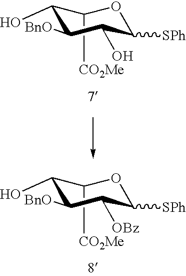

According to an aspect of the present invention there is provided compound 8

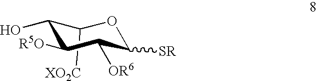

wherein R is an optionally substituted aryl group or an optionally substituted saturated or unsaturated alkyl group; R⁵ and R⁶ are each independently the same or different protecting groups; and X is selected from the group consisting of hydrogen, alkyl and amino. It is preferred that R⁶ is a benzoyl group.

Another aspect of the present invention provides compound 8′

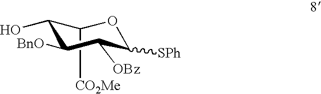

As set out above, important disaccharide building block 10 can be produced by reacting monosaccharides 8 and 9, and monosaccharide 8 can be produced by adding a protecting group to the 2-O atom of compound 7. A further aspect of the present invention relates to a process for the production of compound 7 from compound 6 as shown below

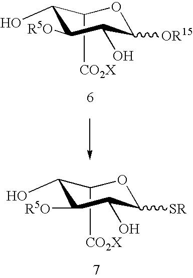

wherein R is an optionally substituted aryl group or an optionally substituted saturated or unsaturated alkyl group; R⁵ is a protecting group; R¹⁵ is an alkyl group; and X is selected from the group consisting of hydrogen, alkyl and amino. R¹⁵ can be an alkyl group or an aryl group, and is preferably a lower alkyl group, such as a methyl group. X may be any type of alkyl group, and is preferably a methyl group.

Preferably conversion of compound 6 to compound 7 is effected by the reaction of compound 6 with a thioalkyl or thioaryl compound (e.g. thiophenol) optionally in the presence of a promotor species (e.g. boron trifluoride diethyl ether complex, BF₃.OEt₂) and optionally a drying agent. The reaction can employ molecular sieves of any appropriate size, with a sieve size of around 4 Å having been found to be particularly suitable. While the inventors do not wish to be bound by any particular theory, it is postulated that the slightly basic nature of the molecular sieves may provide a minor catalytic effect to the conversion of compound 6 to compound 7. While the reaction can be carried out at any suitable temperature, it has been determined that a particularly preferred reaction temperature is around room temperature (r.t.).

A related aspect of the present invention provides compound 7

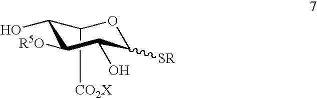

wherein R is an optionally substituted aryl group or an optionally substituted saturated or unsaturated alkyl group; R⁵ is a protecting group; and X is selected from the group consisting of hydrogen, alkyl and amino.

A still further aspect of the present invention provides compound 7′,

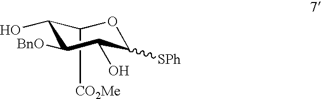

While important monosaccharide 8 may be produced from compound 7 as described above, there is further provided a process for the production of compound 8 from compound 6 below

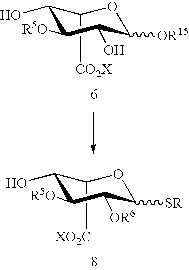

wherein R is an optionally substituted aryl group or an optionally substituted saturated or unsaturated alkyl group; R⁵ and R⁶ are each independently the same or different protecting groups; R¹⁵ is an alkyl group; and X is selected from the group consisting of hydrogen, alkyl and amino. Each of R⁵ and R⁶ may independently be an alkyl or aryl protecting group. Preferably R⁶ is a benzoyl group. R⁵ is preferably an aryl group, such as a benzyl group. In a preferred embodiment compound 8 has the formula 8′

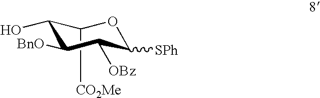

With regard to the above-defined aspect of the present invention conversion of compound 6 to compound 8 is preferably effected by the addition of an appropriate thioalkyl or thioaryl compound, such as thiophenol, and a suitable R⁶-containing compound. For example, when R⁶ is a benzoyl group, as in the above-preferred embodiment, the R⁶-containing compound is preferably a benzoyl halide compound, such as BzCl. The addition of the thioalkyl or thioaryl compound (e.g. thiophenol) may be effected in the presence of a promotor species and/or said addition of the R⁶-containing compound (e.g. benzoyl halide, BzCl) may be effected in the presence of a further promotor species.

According to another aspect of the present invention there is provided a process for the production of compound 8 from compound 3 as shown below

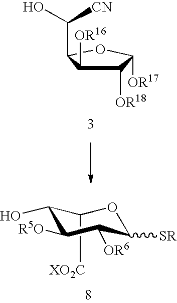

wherein R is an optionally substituted aryl group or an optionally substituted saturated or unsaturated alkyl group; R⁵, R⁶, R¹⁶, R¹⁷ and R¹⁸ are each independently the same or different protecting groups, in which R¹⁷ and R¹⁸ may be linked; and X is selected from the group consisting of hydrogen, alkyl and amino.

R¹⁷ and R¹⁸ may be linked so as to represent an isopropyl group interlinking the 1-O and 2-O atoms of the cyanohydrin compound 3.

R¹⁶ can be an alkyl or aryl protecting group, with a particularly preferred protecting group being a benzyl group.

Independently, R⁵ and R⁶ may each be an alkyl or aryl protecting group. Preferably R⁶ is a benzoyl group and R⁵ is preferably an aryl group, such as a benzyl group.

X is preferably an alkyl group such that the carboxylate moiety (—CO₂X) is an ester group, with methyl being the most preferred option for group X.

With regard to the above defined process for the generation of compound 8 from compound 3 it is preferred that it said reaction is effected by reacting compound 3 with: i) an acetyl halide compound and an alcohol or thionyl chloride (SOCl₂) and a further alcohol, at an appropriate temperature, most specifically around 55° C.; ii) appropriate thioalkyl/thioaryl (e.g. thiophenol (PhSH)); and iii) an R⁶-containing compound, such as benzoyl halide.

Said addition of acetyl halide (e.g. AcCl) and alcohol (e.g. MeOH) is preferably effected at around 55° C.

The addition of the thioalkyl/thioaryl compound (e.g. PhSH) is preferably effected in the presence of a promotor species.

Addition of the R⁶-containing compound (e.g. BzCl) may be effected in the presence of a further promotor species.

Important disaccharide building block 10 can be produced by reacting monosaccharides 8 and 9, as set out above. While various methods for preparing monosaccharide 8 are presented above, there is now presented a further aspect of the present invention which relates to a process for the production of compound 9 from compound 11

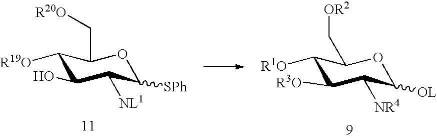

wherein R¹ to R⁴, R¹⁹ and R²⁰ are each independently the same or different protecting groups, R¹⁹ and R²⁰ may be linked; and L and L¹ are each independently the same or different leaving group.

Leaving group, L, may be any appropriate chemical group, and a particularly preferred option in this regard is a trichloroacetimidate (TCA) group.

With regard to the above defined process for the production of compound 9, it is preferred that compound 9 has formula 9′ as shown below

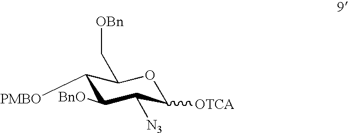

Preferably compound 11 has formula 11′ as shown below

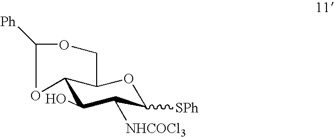

As set out above in respect of the first aspect of the present invention, important disaccharide building block 10 can be coupled to saccharide 21 to yield polysaccharide 20. In connection with this process, saccharide 21 may be a monosaccharide or a polysaccharide depending upon the nature of group Y. In the second preferred embodiment of the first aspect of the present invention set out above in which Y is a monosaccharide unit of formula 22, a preferred embodiment of disaccharide 21 is disaccharide 14, which, according to another aspect of the present invention, may be produced by reacting compound 24 (which is analogous to compound 9 and may be produced in a similar manner as compound 9 as set out above) with a precursor of monosaccharide unit 22, compound 13, as follows

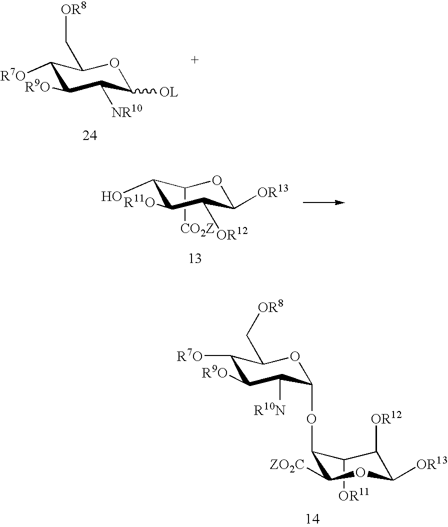

wherein R⁷ to R¹³ are each independently the same or different protecting groups; L is a leaving group; and Z is selected from the group consisting of hydrogen, alkyl and amino. Said leaving group, L, may be any suitable type of chemical group, and is preferably a trichloroacetimidate (TCA) group.

Reaction of compound 24 with compound 13 is preferably effected in the presence of a coupling promotor, such as trimethylsilyl trifluoromethane sulfonate (TMSOTf). The reaction can be effected at any appropriate temperature. Suitable temperatures are generally below around 0° C., and a preferred reaction temperature is around −50° C.

Compound 13 used above to prepare disaccharide building block 14 may be produced from compound 25 (which is analogous to compound 6 above) as follows

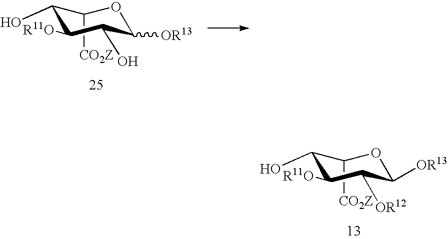

wherein R¹¹ and R¹³ are each independently the same or different protecting groups; and Z is selected from the group consisting of hydrogen, alkyl and amino.

R¹¹ and R¹³ may be an alkyl group or an aryl group. R¹¹ is preferably an aryl group, such as a benzyl group. R¹² is preferably an aryl group, such as a benzoyl group. R¹³ is preferably an alkyl group, such as a methyl group.

Z is preferably an alkyl group such that the carboxylate moiety is an ester group, a preferred alkyl group being a methyl group.

Conversion of compound 25 to compound 13 by addition of the R¹² protecting group may be effected by the reaction of compound 25 with a benzoyl halide, such as BzCl to provide a 2-O benzoyl protecting group.

Once desired polysaccharides have been produced by appropriate coupling chemistry as presented above, it is preferred that the polysaccharides thus formed be at least partially, more preferably fully, sulfated so that they can be employed in a biological context and subjected to biological testing. Accordingly, in a further aspect of the present invention, generic polysaccharide 20 is converted to fully polysaccharide 27 as shown below

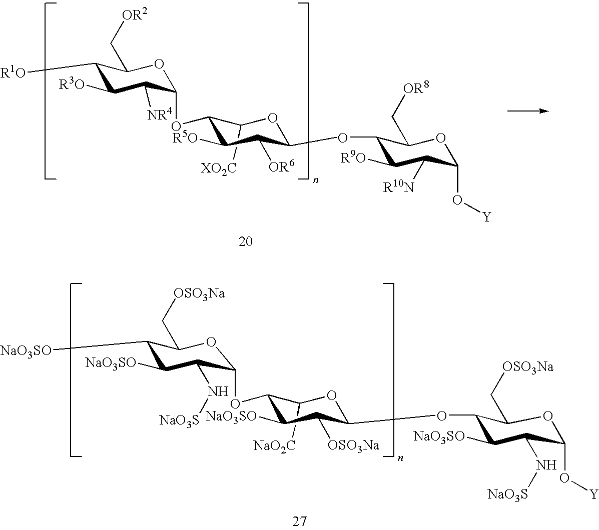

wherein R¹ to R⁶ and R⁸ to R¹⁰ are each independently the same or different protecting groups; X is selected from the group consisting of hydrogen, alkyl and amino; Y is selected from the group consisting of a protecting group and one or more saccharide residues; and n is a positive integer.

The above aspect, may be considered to comprise at least two steps, a first partial sulfation step followed by a second step to fully sulfate the polysaccharide. Thus, a further aspect of the present invention provides a process for the conversion of generic polysaccharide 20 to partly sulfated polysaccharide 26 as shown below

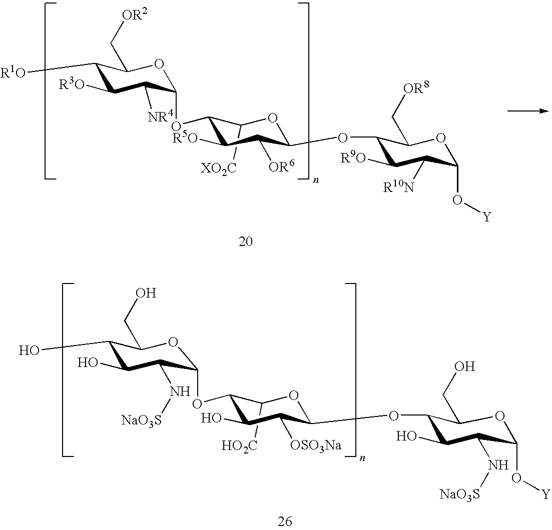

wherein R¹ to R⁶ and R⁸ to R¹⁰ are each independently the same or different protecting groups; X is selected from the group consisting of hydrogen, alkyl and amino; Y is selected from the group consisting of a protecting group and one or more saccharide residues; and n is a positive integer.

Another aspect provides for further sulfation of partly sulfated polysaccharide 26 to fully sulfated polysaccharide 27 as follows

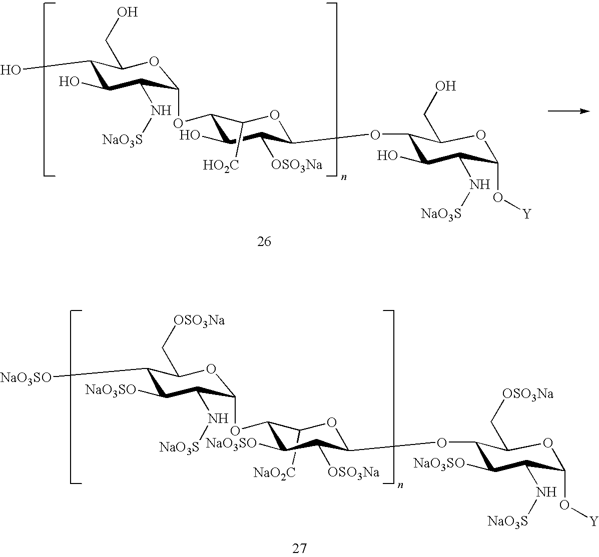

wherein Y is selected from the group consisting of a protecting group and one or more saccharide residues; and n is a positive integer.

In one or more of the above three aspects of the present invention R⁶ is preferably a benzoyl group.

As discussed above in the context of the first aspect of the present wherein polysaccharide 20 is produced by reacting compounds 10 and 21, in a first preferred embodiment of the first aspect, group Y in compound 21 is R¹⁴, which provides preferred compound 12, which, following removal of its R⁷ protecting group, can be coupled to compound 10 to provide a first preferred polysaccharide 15 (which represents a first preferred embodiment of polysaccharide 20). A further aspect of the present invention, which also represents a first preferred embodiment of the aspect set out above wherein polysaccharide 20 is converted to polysaccharide 26, provides a process for the conversion of polysaccharide 15 to polysaccharide 17 as shown below

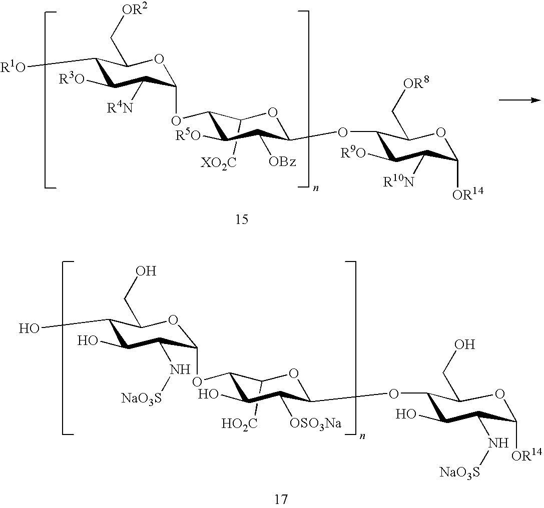

wherein R¹ to R⁵, R⁸ to R¹⁰ are each independently the same or different protecting groups; R¹⁴ is a substituted or unsubstituted aryl group or a substituted or unsubstituted saturated or unsaturated alkyl group; X is selected from the group consisting of hydrogen, alkyl and amino; and n is a positive integer.

It will be appreciated that polysaccharides 15, 17 and 19 described herein represent first preferred embodiments of polysaccharides 20, 26 and 27 respectively. As such, the discussion below of preferred features of polysaccharides 15, 17 and 19, and of the methods for converting polysaccharide 15 to 17 and then 19, are equally applicable to polysaccharides 20, 26 and 27 respectively and to the process for converting polysaccharide 20 to 26 and then 27.

With regard to the above defined aspects of the present invention relating to sulfation of polysaccharides, R¹ may be any appropriate protecting group, such as a PMB group. At least one, preferably all of R², R³, R⁵, R⁹ and R⁸ are alkyl or aryl groups, more preferably aryl groups and most preferably benzyl groups. The two nitrogen containing groups, NR⁴ and NR¹⁰ are preferably azide groups, such as N₃ groups. X in compound 15 may be any appropriate alkyl group, such as a methyl group.

Said conversion of compound 15 to the partly sulfated analogue, compound 17 preferably involves replacement of X with a first hydrogen atom and replacement of the 2-O benzoyl group with a second hydrogen atom. Said replacement process may employ the addition of LiOOH at around room temperature (r.t.). Sulfation of the 2-O may then be achieved using a suitable sulfation reagent, and a preferred compound is Py.SO₃ complex.

It is preferred that the conversion of compound 15 to compound 17 further involves replacement of at least one, more preferably the majority, and most preferably all of protecting groups R¹, R², R³, R⁵, R⁹, R⁸ with hydrogen atoms. This may be achieved in any desirable manner, a preferred option being to employ hydrogenolysis (e.g. using Pd(OH)₂/H₂ although any suitable hydrogenolysis procedure may be employed) at ambient or around room temperature (r.t.) or higher, for example around 50 to 55° C. The hydrogenolysis reaction in may be conducted in MeOH/H₂O (between 1:1 and 4:1 MeOH:H₂O). This conversation process also concurrently effects reduction of one or most preferably both NR⁴ and NR¹⁰ to primary amino groups, such as —NH₂ primary amino groups. Sulfation of the amino groups and added hydrogen atoms may then be effected. Thus, the amino groups derived from the NR⁴ and NR¹⁰ groups may be sulfated. Any suitable sulfation reagent may be employed, and a preferred compound is Py.SO₃ complex, which may be added at around room temperature (r.t.) or at a higher temperature, for example around 50 to 55° C.

Once the partly sulfated polysaccharide 17 has been produced according to the above defined aspect of the present invention, a process representing a yet further aspect of the present invention provides the conversion of compound 17 to its fully sulfated analogue, compound 19 below

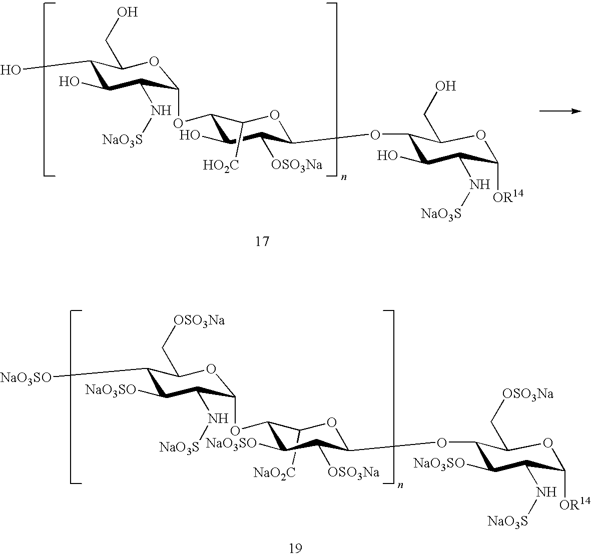

wherein R¹⁴ is a substituted or unsubstituted aryl group or a substituted or unsubstituted saturated or unsaturated alkyl group; and n is a positive integer. R¹⁴ may be an alkyl or aryl group, and is preferably an alkyl group, such as a methyl group.

Conversion of compound 17 to compound 19 preferably employs the addition of TMA.SO₃ at a temperature of around −20 to 0° C.

As hereinbefore described, in an alternative preferred embodiment of the first aspect of the present invention, group Y in compound 21 is the monosaccharide unit 22 which provides preferred disaccharide 14, which, following removal of its R⁷ protecting group, can be coupled to compound 10 to provide a second preferred polysaccharide 16 (which represents a second preferred embodiment of polysaccharide 20). A further aspect of the present invention, which also represents a second preferred embodiment of the aspect set out above wherein polysaccharide 20 is converted to polysaccharide 26, provides a process for the conversion of polysaccharide 16 to polysaccharide 18 as shown below

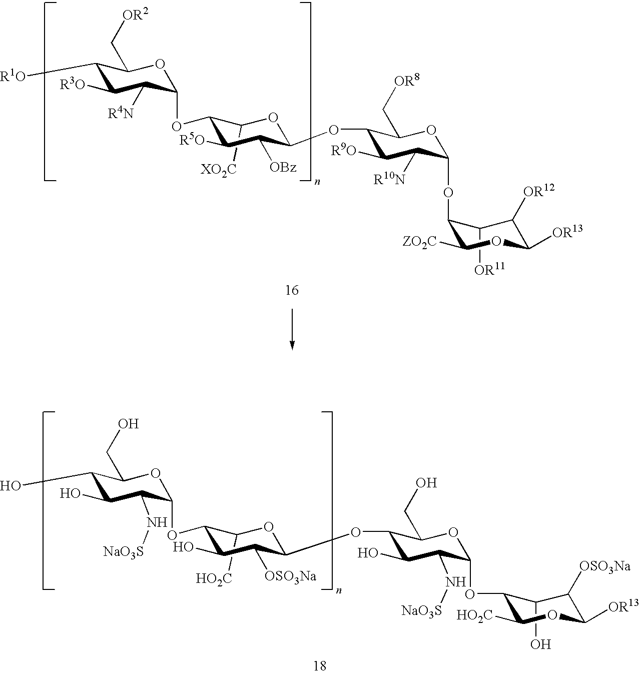

wherein R¹ to R⁵, R⁸ to R¹⁰ are each independently the same or different protecting groups; R¹⁴ is a substituted or unsubstituted aryl group or a substituted or unsubstituted saturated or unsaturated alkyl group; X is selected from the group consisting of hydrogen, alkyl and amino; and n is a positive integer.

It will be appreciated that polysaccharides 16, 18 and 23 described herein represent second preferred embodiments of polysaccharides 20, 26 and 27 respectively. As such, the discussion below of preferred features of polysaccharides 16, 18 and 23, and of the methods for converting polysaccharide 16 to 18 and then 23, are equally applicable to polysaccharides 20, 26 and 27 respectively and to the process for converting polysaccharide 20 to 26 and then 27 as set out above.

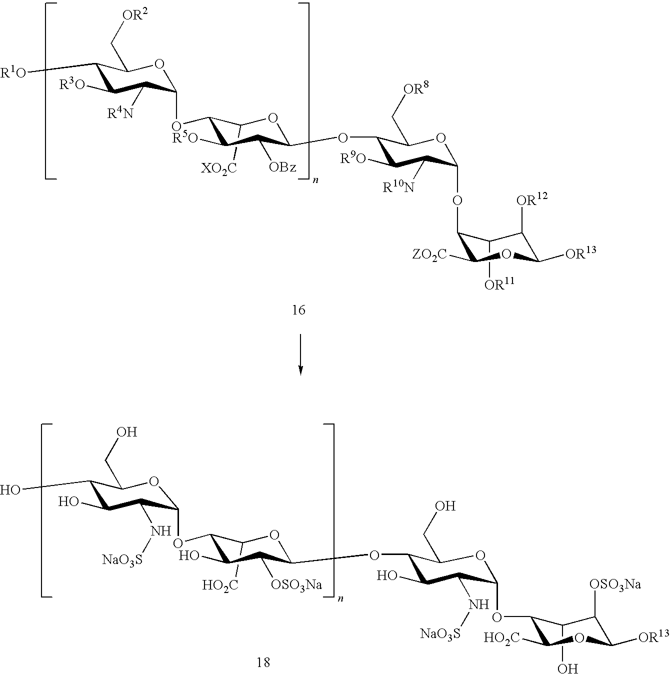

wherein R¹ to R⁵, R⁸ to R¹³ are each independently the same or different protecting groups; X and Z are each independently selected from the group consisting of hydrogen, alkyl and amino; and n is a positive integer.

R¹ may be any suitable protecting group, such as a PMB group. At least one, more preferably all of R², R³, R⁵, R⁹, R⁸, and R¹¹ are alkyl or aryl groups, a preferred option being benzyl groups. NR⁴ and/or NR¹⁰ are/is preferably an azide group, such as N₃. X and Z may each independently be any form of alkyl group, such as a methyl group.

Conversion of unsulfated compound 16 to partly sulfated compound 18 may comprise replacement of X and Z with first and second hydrogen atoms respectively and/or replacement of the 2-O benzoyl group with a third hydrogen atom. Said replacement process preferably employs the addition of LiOOH at around room temperature (r.t.). Sulfation of the 2-O may then be achieved using a suitable sulfation reagent, and a preferred compound is Py.SO₃ complex.

Conversion of compound 16 to compound 18 may further involve replacement of at least one, more preferably the majority, and most preferably all, of protecting group R¹, R², R³, R⁵, R⁹, R⁸, R¹¹ with hydrogen atoms. Said replacement process may employ hydrogenolysis (e.g. using Pd(OH)₂/H₂ although any suitable hydrogenolysis procedure may be employed) at around room temperature (r.t.) or higher, for example around 50 to 55° C. The hydrogenolysis reaction in may be conducted in MeOH/H₂O (between 1:1 and 4:1 MeOH:H₂O). Moreover, said conversation process may involve reduction of NR⁴ and/or NR¹⁰ to amino groups, preferably primary —NH₂ amino groups. The amino groups may be sulfated and said first, second and third hydrogen atoms may be converted to sulfate groups to yield the partly sulfated polysaccharide 18. Selective sulfation of the amino groups and the first, second and third hydrogen atoms may employ any desirable sulfating agent, and preferably employs the addition of Py.SO₃ or TMA.SO₃ complex at around room temperature (r.t.) more preferably at a temperature of around 50° C. to 55° C.

As mentioned above, the partly sulfated polysaccharide 18 can be converted to its fully sulfated analogue, compound 23 as shown below, this process representing a further aspect of the present invention.

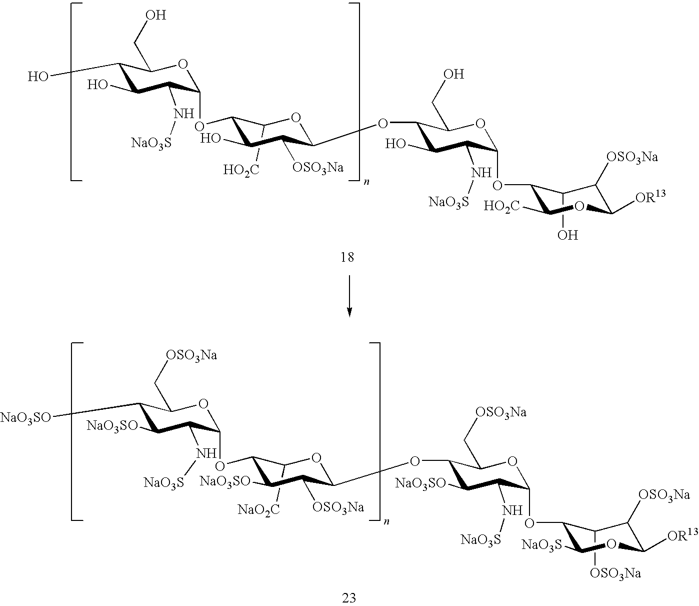

wherein R¹³ is a protecting group; and n is a positive integer. R¹³ may be an alkyl or aryl group, and is preferably an alkyl group, such as a methyl group. Conversion of compound 18 to compound 23 preferably employs the addition of TMA.SO₃ at a temperature of around −20 to 0° C.

### EXAMPLE

Methods are described below to produce partially and fully sulfated polysaccharides starting from α-D-glucose via potentially valuable intermediate compounds. The methods can be used to generate odd or even numbered polysaccharides by the addition of a mono- or di-saccharide acceptor compound to a thioglycoside donor compound. Aspects of the present invention relate to various steps in the methods set out below and various intermediate compounds.

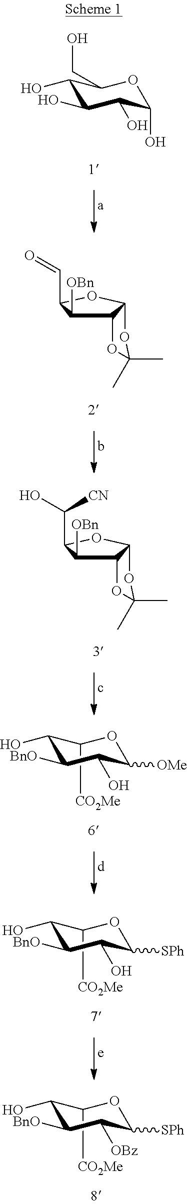

The reaction conditions employed in respect of each step of Scheme 1 are set out below.
Step a) comprises (i) ZnCl₂, I₂, 16 hrs at r.t. then 5 h at reflux; (ii) BnCl, Bu₄NHSO₄, NaOH in H₂O, THF, 3 h at reflux; (iii) AcOH, H₂O, 6 h at 60° C.; (iv) NaIO₄, EtOH, H₂O, 2 h at r.t.
Step b) comprises KCN, MgCl₂.6H₂O, EtOH (dil.), 5 days, r.t. Yield=71% (from step a(ii))
Step c) comprises AcCl, MeOH, 16 h, 50-55° C., 86%.
Step d) comprises PhSH, BF₃.OEt₂, 4 Å sieves, DCM, 90 min, r.t.
Step e) comprises Bu₂SnO, MeOH, 1 h, reflux, then BzCl, toluene, 30 min, 0° C. Yield=55%.

Compound 8′ can then be converted to disaccharide building block 10′ as follows.

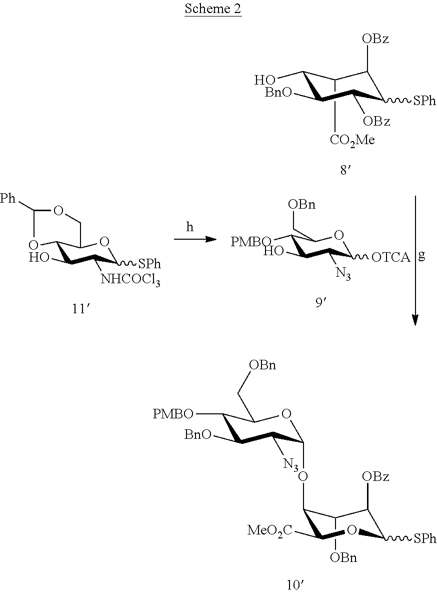

The reaction conditions employed in respect of each step of Scheme 2 are set out below.
Step g) comprises TMSOTf, DCM, 1 h, −20 to −30° C. Yield=58%.
Step h) comprises:
   h1) KOH (3 eq.), H₂O/MeOH/THF, r.t., 3 h;
   h2) (ImSO₂N₃).HCl, MeOH, K₂CO₃, r.t., 12 h;
   h3) BnBr, NaH, THF, reflux, 5 h. Yield=88%;
   h4) Et₃SiH, BF₃.OEt₂, DCM, 0° C., 2 h. Yield=95%;
   h5) PMBCl, NaH, THF, reflux, 2 h. Yield=90%;
   h6) NBS (1.1 eq.) in acetone, 0° C., 45 mins. Yield=95%; and
   h7) CCl₃CN, DBU (catalytic), DCM, 90 mins. Yield=99%.

A method for preparing a preferred embodiment of a further disaccharide building block 14′, which can be coupled to compound 10′, is set out below.

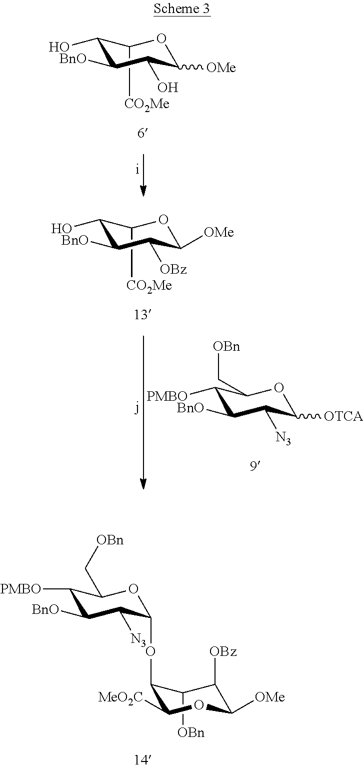

The reaction conditions employed in respect of each step of Scheme 3 are set out below.
Step i) comprises Bu₂SnO, MeOH, 1 to 1.5 h, reflux then BzCl, toluene, 30 min, 0° C. Yield=55%. Note: A higher temperature (e.g. around 55° C.) can be used for step (i).
Step j) comprises TMSOTf, DCM, 1 h, −50° C. Yield=70%.

In a preferred embodiment of the first aspect of the present invention monosaccharide 12′ (a preferred embodiment of the saccharide of general formula 21 which forms part of the definition of the first aspect of the present invention) is first deprotected to provide deprotected monosaccharide 12A′ followed by coupling to disaccharide building block 10′ as shown below. Following the coupling of one unit of compound 10′ to one unit of compound 12A′ to produce polysaccharide 15′ in which n=1, any desirable number of further units of compound 10′ can be added repeating the following two steps any desirable number of times: i) removing the terminal PMB protecting group of polysaccharide 15′; and ii) adding one further unit of compound 10′, as shown below in respect of step k,

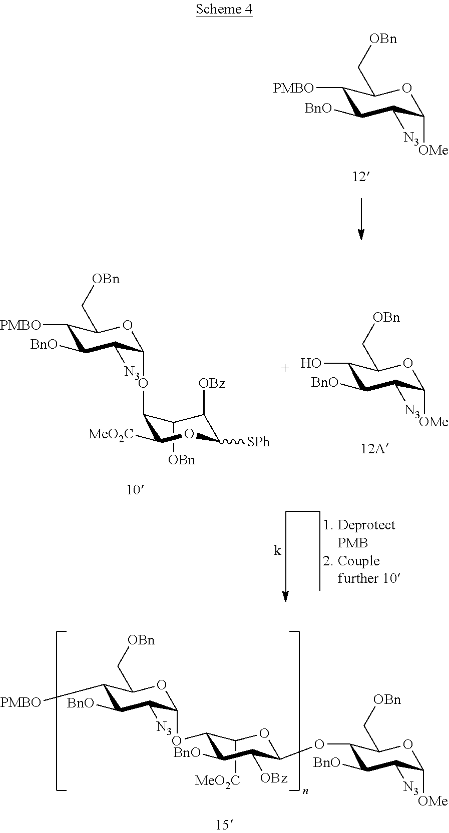

The reaction conditions employed in respect of Scheme 4 are set out below.
Step k) comprises:
   k1) DDQ, DCM/H₂O 9:1, H₂O, 1.5 h, r.t. Yield=76-85%; and
   k2) NIS, AgOTf, DCM, 30 min, 0° C. Yield=83-89%.

In a preferred embodiment of the first aspect of the present invention monosaccharide 14′ (a preferred embodiment of the saccharide of general formula 21 which forms part of the definition of the first aspect of the present invention) is first deprotected to provide deprotected monosaccharide 14A′ followed by coupling to disaccharide building block 10′ as shown below. Following the coupling of one unit of compound 10′ to one unit of compound 14A′ to produce polysaccharide 16′ in which n=1, any desirable number of further units of compound 10′ can be added repeating the following two steps any desirable number of times: i) removing the terminal PMB protecting group of polysaccharide 16′; and ii) adding one further unit of compound 10′, as shown below in respect of step k,

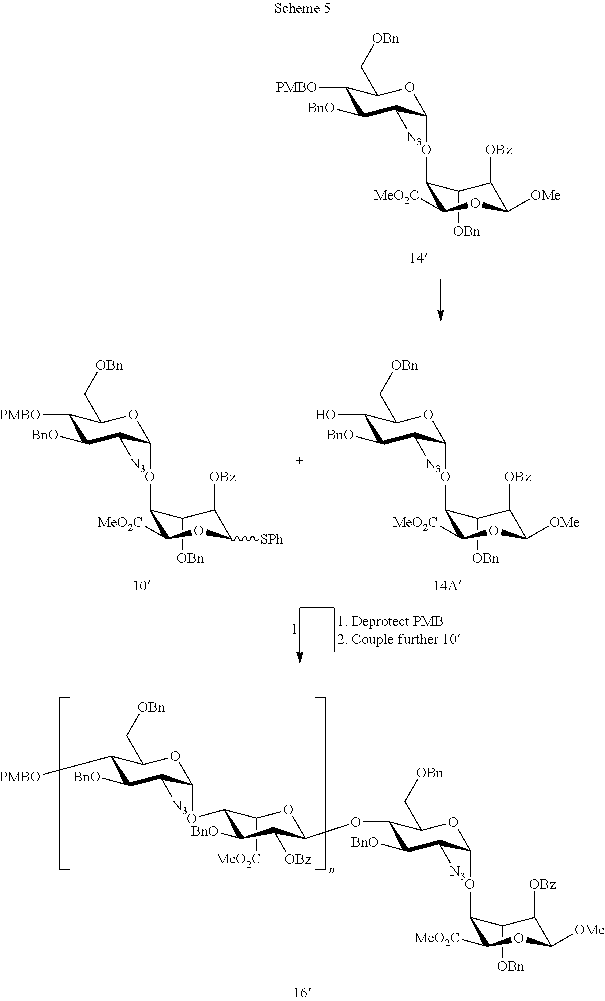

The reaction conditions employed in respect of Scheme 5 are set out below.
Step l) comprises:
   l1) CAN, MeCN, H₂O, 3 h, r.t. Yield=77-81%; and
   l2) NIS, AgOTf, DCM, 30 min, 0° C. Yield=73-94%.

Polysaccharide 15′ prepared in Scheme 4 above can be selectively sulfated as follows to provide partly sulfated polysaccharide 17′, which incorporates sulfate groups at the 2-0 and 2-N positions only.

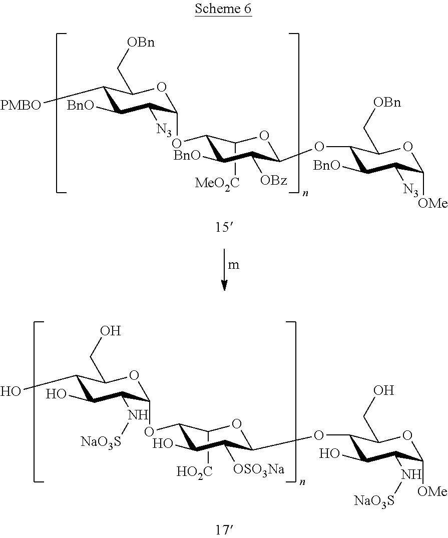

The reaction conditions employed in respect of Scheme 6 are set out below.
Step m) comprises:
   m1) LiOOH, THF, H₂O, 24 hrs, r.t. then KOH, MeOH, 24 h, r.t. Yield=62-77%;
   m2) SO₃.Py, Py, 2 h, r.t. 50-55° C. Yield=80%; and
   m3) H₂/Pd(OH)₂ in MeOH/H₂O (between 1:1 and 4:1 MeOH:H₂O), up to 48 h (e.g. 18 h), ambient temperature (higher temperatures, e.g. 50-55° C. can be used). Yield=99-100%.
   m4) Excess SO₃.Py in water, NaHCO₃, 24 h, rt, Yield=80-85%.

Polysaccharide 16′ prepared in Scheme 5 above, can be selectively sulfated as follows to provide partly sulfated polysaccharide 18′, which incorporates sulfate groups at the 2-O and 2-N positions only.

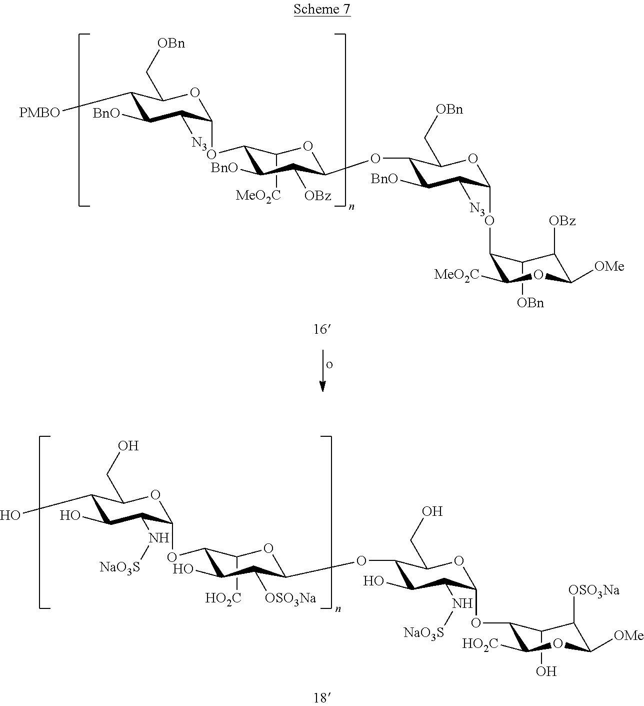

The reaction conditions employed in respect of Scheme 7 are set out below.
Step o) comprises:
   o1) LiOOH, THF, H₂O, 24 h, r.t. then KOH, MeOH, 24 h, r.t. Yield=58-62%;
   o2) SO₃.Py, Py, 2 h, r.t. 50-55° C. Yield=80%; and
   o3) H₂/Pd(OH)₂ in MeOH/H₂O (between 1:1 and 4:1 MeOH:H₂O), up to 48 h (e.g. 18 h), ambient temperature (higher temperatures, e.g. 50-55° C. can be used). Yield=99-100%.
   o4) Excess SO₃.Py in water, NaHCO₃, 24 h, rt, Yield=80-85%.

Partly sulfated polysaccharide 17′ prepared in Scheme 6 above, can be fully sulfated as follows to provide fully sulfated polysaccharide 19′, which incorporates sulfate groups at the 2-O, 3-O, 6-O and 2-N positions only.

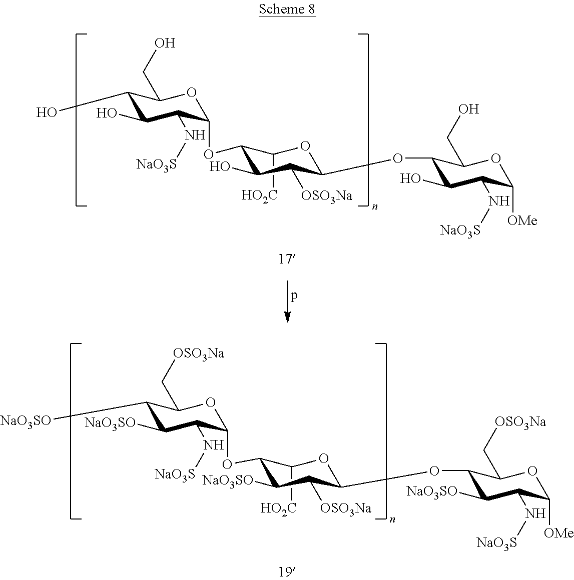

The reaction conditions employed in respect of Scheme 8 are set out below.
Step p) comprises:
   p1) Excess TMA.SO₃, triflic acid, DMF, −20° to 0° C., 24 h. Yield=62-77%

Partly sulfated polysaccharide 18′ prepared in Scheme 7 above, can be fully sulfated as follows to provide fully sulfated polysaccharide 23′, which incorporates sulfate groups at the 2-O, 3-O, 6-O and 2-N positions only.

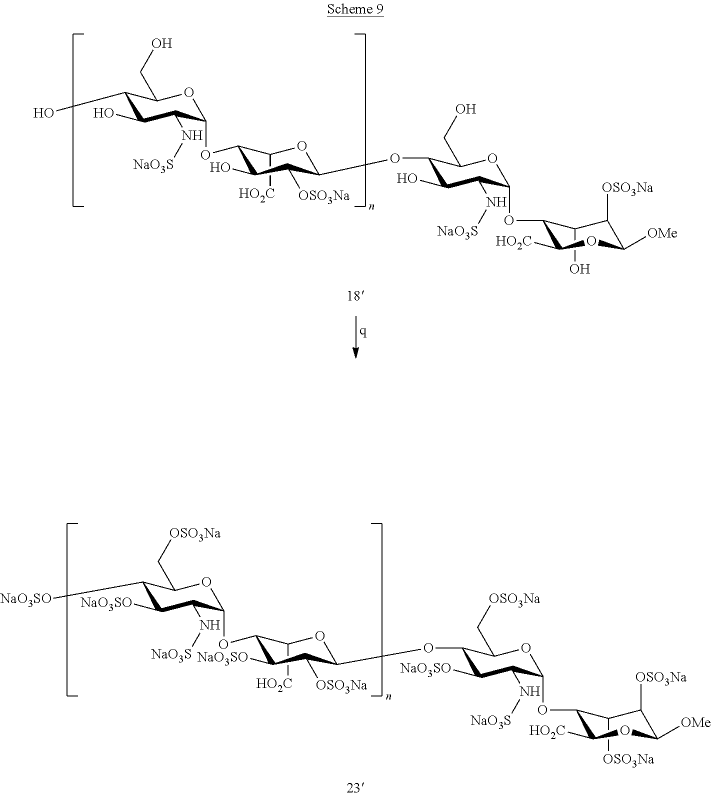

The reaction conditions employed in respect of Scheme 9 are set out below.
Step q) comprises:
   q1) TMA.SO₃, DMF, heat at 50° C., 24 h,

### REFERENCES

1. Board, R. E., et al. Stage- and CAl25-related survival in patients with epithelial ovarian cancer treated at a cancer center. Int J Gynecol Cancer 16 Suppl 1, 18-24 (2006).
2. Rosa, D. D., et al. The neoadjuvant approach in the treatment of patients with advanced epithelial ovarian carcinoma. Clinical Oncology (Royal College of Radiologists (Great Britain)) 19, 125-128 (2007).
3. Rosa, D. D., et al. The interval from surgery to chemotherapy in the treatment of advanced epithelial ovarian carcinoma. Eur J Surg Oncol 32, 588-591 (2006).
4. Whitworth, M. K., et al. Regulation of fibroblast growth factor-2 activity by human ovarian cancer tumor endothelium. Clin Cancer Res 11, 4282-4288 (2005).
5. Narita, K., et al. HSulf-1 inhibits angiogenesis and tumorigenesis in vivo. Cancer Research 66, 6025-6032 (2006).
6. Gan, Y., Wientjes, M. G. & Au, J. L. Expression of basic fibroblast growth factor correlates with resistance to paclitaxel in human patient tumors. Pharmaceutical Research 23, 1324-1331 (2006).
7. Steele, I. A., Edmondson, R. J., Leung, H. Y. & Davies, B. R. Ligands to FGF receptor 2-IIIb induce proliferation, motility, protection from cell death and cytoskeletal rearrangements in epithelial ovarian cancer cell lines. Growth Factors (Chur, Switzerland) 24, 45-53 (2006).
8. Steele, I. A., et al. Induction of FGF receptor 2-IIIb expression and response to its ligands in epithelial ovarian cancer. Oncogene 20, 5878-5887 (2001).
9. Valve, E., et al. Expression of fibroblast growth factor (FGF)-8 isoforms and FGF receptors in human ovarian tumors. Int J Cancer 88, 718-725 (2000).
10. Schmitt, J. F., Susil, B. J. & Hearn, M. T. Aberrant FGF-2, FGF-3, FGF-4 and C-erb-B2 gene copy number in human ovarian, breast and endometrial tumours. Growth Factors (Chur, Switzerland) 13, 19-35 (1996).
11. Rosen, A., et al. First experience with FGF-3 (INT-2) amplification in women with epithelial ovarian cancer. Br J Cancer 67, 1122-1125 (1993).
12. Casanovas, O., Hicklin, D. J., Bergers, G. & Hanahan, D. Drug resistance by evasion of antiangiogenic targeting of VEGF signaling in late-stage pancreatic islet tumors. Cancer Cell 8, 299-309 (2005).
13. Batchelor, T. T., et al. AZD2171, a pan-VEGF receptor tyrosine kinase inhibitor, ormalizes tumor vasculature and alleviates edema in glioblastoma patients. Cancer Cell 11, 83-95 (2007).
14. Ornitz, D. M., et al. Receptor Specificity of the Fibroblast Growth Factor Family. J. Biol. Chem. 271, 15292-15297 (1996).
15. Yasumoto, H., Matsubara, A., Mutaguchi, K., Usui, T. & McKeehan, W. L. Restoration of fibroblast growth factor receptor2 suppresses growth and tumorigenicity of malignant human prostate carcinoma PC-3 cells. Prostate 61, 236-242 (2004).
16. Carstens, R. P., Eaton, J. V., Krigman, H. R., Walther, P. J. & Garcia-Blanco, M. A. Alternative splicing of fibroblast growth factor receptor 2 (FGF-R2) in human prostate cancer. Oncogene 15, 3059-3065 (1997).
17. Yayon, A., Klagsbrun, M., Esko, J. D., Leder, P. & Ornitz, D. M. Cell surface, heparin-like molecules are required for binding of basic fibroblast growth factor to its high affinity receptor. Cell 64, 841-848 (1991).
18. Pye, D. A., Vives, R. R., Turnbull, J. E., Hyde, P. & Gallagher, J. T. Heparan sulfate oligosaccharides require 6-Osulphation for promotion of basic fibroblast growth factor mitogenic activity. J Biol Chem 273, 22936-22942 (1998).
19. Kohl, R., Antoine, M., Reimers, K. & Kiefer, P. FGF3 Attached to a Phosholipid Membrane Anchor Gains a High Transforming Capacity. Implications of microdomains for FGF3 cell transformation. 277 32760-32767 (2002).
20. Luo, Y., Ye, S., Kan, M. & McKeehan, W. L. Control of FGF7- and FGF1-induced mitogenesis and downstream signaling by distinct heparin octasaccharide motifs. J Biol Chem, In press (2006).
21. Luo, Y., Ye, S., Kan, M. & McKeehan, W. L. Structural specificity in a FGF7-affinity purified heparin octasaccharide required for formation of a complex with FGF7 and FGFR2IIIb. J Cell Biochem 97, 1241-1258 (2006).
22. Jayson, G. C. & Gallagher, J. T. Heparin oligosaccharides: inhibitors of the biological activity of bFGF on Caco-2 cells. Br J Cancer 75, 9-16 (1997).
23. Hasan, J., et al. Heparin Octasaccharides Inhibit Angiogenesis In vivo. Clin Cancer Res 11, 8172-8179 (2005).
24. Raman, R., Venkataraman, G., Ernst, S., Sasisekharan, V. & Sasisekharan, R. Structural specificity of heparin binding in the fibroblast growth factor family of proteins. PNAS 100, 2357-2362 (2003).
25. Turnbull, J. E., Fernig, D. G., Ke, Y., Wilkinson, M. C. & Gallagher, J. T. Identification of the basic fibroblast growth factor binding sequence in fibroblast heparan sulfate. J Biol Chem 267, 10337-10341 (1992).
26. Lundin, L., et al. Selectively desulfated heparin inhibits fibroblast growth factor-induced mitogenicity and angiogenesis. J Biol Chem 275, 24653-24660 (2000).
27. Ishihara, M., et al. Importance of 6-O-sulfate groups of glucosamine residues in heparin for activation of FGF-1 and FGF-2. J Biochem (Tokyo) 118, 1255-1260 (1995).
28. Rusnati, M., et al. Distinct role of 2-O-, N-, and 6-O-sulfate groups of heparin in the formation of the ternary complex with basic fibroblast growth factor and soluble FGF receptor-1. Biochem Biophys Res Commun 203, 450-458 (1994).
29. Aviezer, D., et al. Differential structural requirements of heparin and heparan sulfate proteoglycans that promote binding of basic fibroblast growth factor to its receptor. J Biol Chem 269, 114-121 (1994).
30. Kan, M., et al. An essential heparin-binding domain in the fibroblast growth factor receptor kinase. Science 259, 1918-1921 (1993).
31. Plotnikov, A. N., Hubbard, S.R., Schlessinger, J. & Mohammadi, M. Crystal structures of two FGF-FGFR complexes reveal the determinants of ligand-receptor specificity. Cell 101, 413-424. (2000).
32. Yeh, B. K., et al. Structural basis by which alternative splicing confers specificity in fibroblast growth factor receptors. Proc Natl Acad Sci U S A 100, 2266-2271. (2003).
33. Robinson, C. J., Harmer, N. J., Goodger, S. J., Blundell, T. L. & Gallagher, J. T. Cooperative Dimerization of Fibroblast Growth Factor 1 (FGF1) upon a Single Heparin Saccharide May Drive the Formation of 2:2:1 FGF1 {middle dot}FGFR2c {middle dot}Heparin Ternary Complexes. J Biol Chem 280, 42274-42282 (2005).
34. Powell, A. K., Fernig, D. G. & Turnbull, J. E. Fibroblast growth factor receptors 1 and 2 interact differently with heparin/heparan sulfate. Implications for dynamic assembly of a ternary signaling complex. J Biol Chem 277, 28554-28563. (2002).
35. Maccarana, M., Casu, B. & Lindahl, U. Minimal sequence in heparin/heparan sulfate required for binding of basic fibroblast growth factor [published erratum appears in J Biol Chem 1994 Feb. 4; 269(5):3903]. J Biol Chem 268, 23898-23905 (1993).
36. Kovensky, J., et al. A synthetic heparan sulfate pentasaccharide, exclusively containing 1-iduronic acid, displays higher affinity for FGF-2 than its D-glucuronic acid-containing isomers. Bioorg Med Chem 7, 1567-1580 (1999).
37. Ostrovsky, O., et al. Differential effects of heparin saccharides on the formation of specific fibroblast growth factor (FGF) and FGF receptor complexes. J Biol Chem 277, 2444-2453. (2002).
38. Basche, M., et al. A Phase I Biological and Pharmacologic Study of the Heparanase Inhibitor PI-88 in Patients with Advanced Solid Tumors. Clinical Cancer Research 12, 5471-5480 (2006).
39. Joyce, J. A., Freeman, C., Meyer-Morse, N., Parish, C. R. & Hanahan, D. A functional heparan sulfate mimetic implicates both heparanase and heparan sulfate in tumor angiogenesis and invasion in a mouse model of multistage cancer. Oncogene 24, 4037-4051 (2005).
40. Parish, C. R., Freeman, C., Brown, K. J., Francis, D. J. & Cowden, W. B. Identification of sulfated oligosaccharide-based inhibitors of tumor growth and metastasis using novel in vitro assays for angiogenesis and heparanase activity. Cancer Research 59, 3433-3441 (1999).

1. A process for the production of polysaccharide 20 from disaccharide 10 and saccharide 21 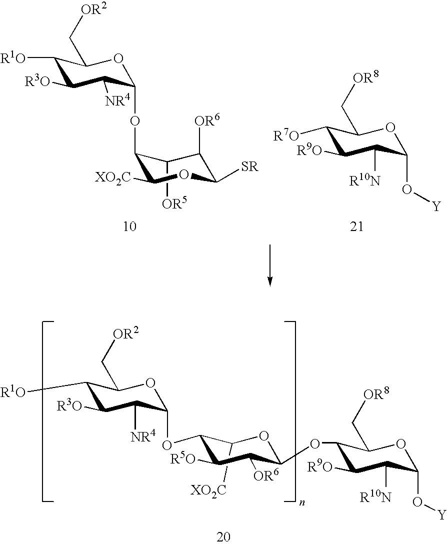
wherein R¹ to R¹⁰ are each independently the same or different protecting groups; R is an optionally substituted aryl group or an optionally substituted saturated or unsaturated alkyl group; X is selected from the group consisting of hydrogen, alkyl and amino; Y is selected from the group consisting of a protecting group and one or more saccharide residues; and n is a positive integer; and
further wherein said process comprises removal of the R⁷ protecting group and reaction of the deprotected C4-oxygen atom of compound 21 with the C1-carbon atom of the 1-ido moiety of compound 10.

2-98. (canceled)

99. A process according to claim 1, wherein at least one of NR⁴ and NR¹⁰ is an azide group.

100. A process according to claim 1, wherein the process further comprises one or more polysaccharide elongation steps, each step comprising removal of the R¹ protecting group of polysaccharide 20 and reaction of the deprotected C4-oxygen atom of polysaccharide 20 with the C1-carbon atom of the 1-ido moiety of a further unit of compound 10, n being increased by one for the or each polysaccharide elongation step.

101. A process according to claim 1, wherein Y is an alkyl group.

102. A process for the production of polysaccharide 15′ from disaccharide 10′ and monosaccharide 12′ 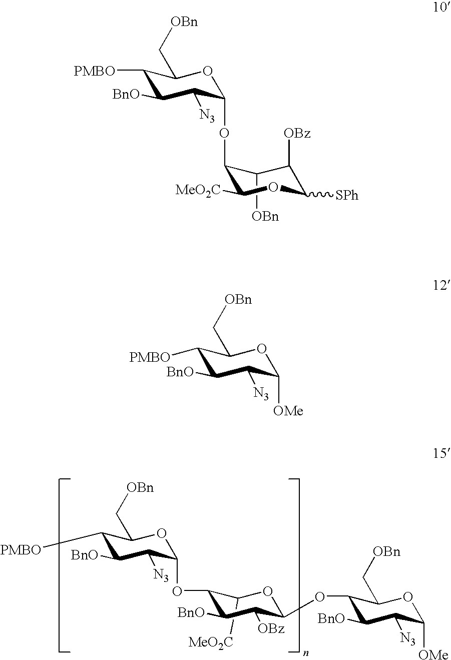
wherein n is a positive integer; and
further wherein said process comprises removal of the PMB protecting group of compound 12′ and reaction of the deprotected C4-oxygen atom of compound 12′ with the C1-carbon atom of the 1-ido moiety of compound 10′.

103. A process according to claim 102, wherein the process further comprises one or more polysaccharide elongation steps, each step comprising removal of the PMB protecting group of polysaccharide 15′ and reaction of the deprotected C4-oxygen atom of polysaccharide 15′ with the C1-carbon atom of the 1-ido moiety of a further unit of compound 10′, n being increased by one for the or each polysaccharide elongation step.

104. A process according to claim 1, wherein Y is a monosaccharide unit 22 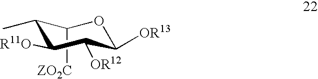
wherein at least one of R¹¹, R¹² and R¹³ is a protecting group; and Z is selected from the group consisting of hydrogen, alkyl and amino.

105. A process for the production of polysaccharide 16′ from disaccharide 10′ and disaccharide 14′ 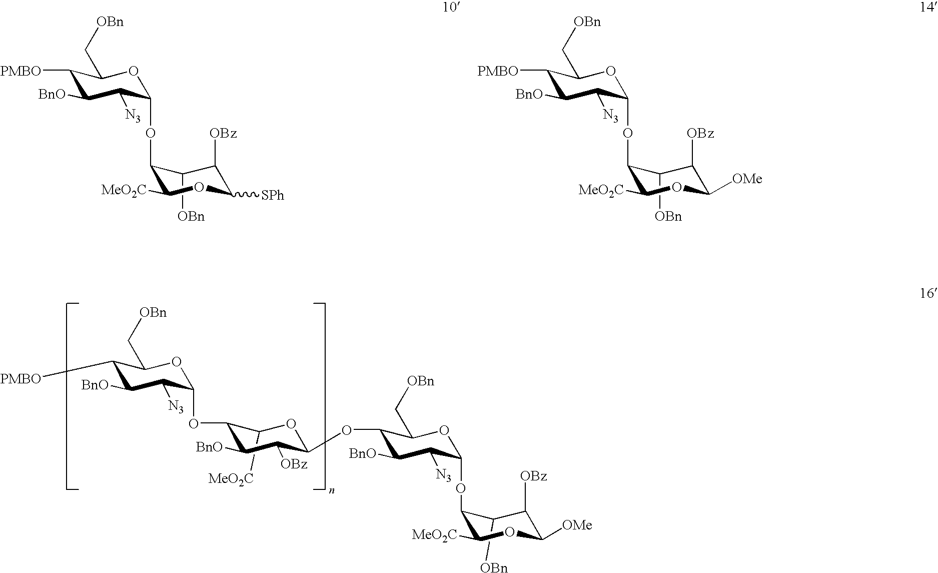
wherein n is a positive integer; and further wherein said process comprises removal of the PMB protecting group of compound 14′ and reaction of the deprotected C4-oxygen atom of the compound 14′ with the C1-carbon atom of the 1-ido moiety of compound 10′.

106. A polysaccharide compound of formula 20′ 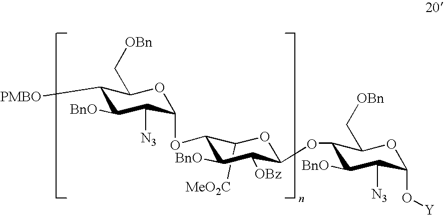
wherein Y is a protecting group or one or more saccharide residue; and n is a positive integer.

107. A polysaccharide 16 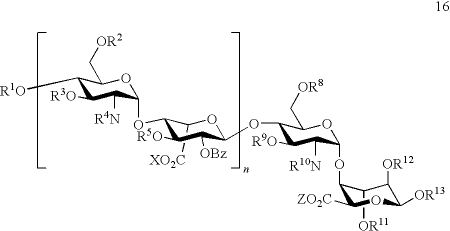
wherein each of R¹ to R⁵, R⁸ to R¹³ are each independently the same or different protecting groups; X and Z are each independently selected from the group consisting of hydrogen, alkyl and amino; and n is a positive integer.

108. A polysaccharide 16′ 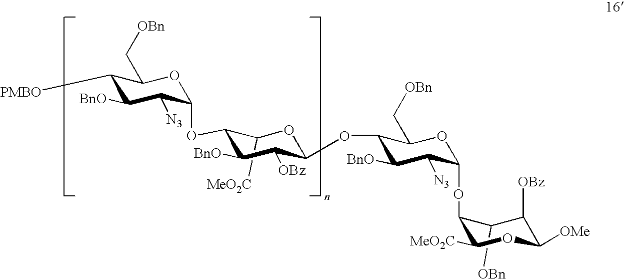
wherein n is a positive integer.

109. A process for the production of disaccharide 10 by the reaction of compound 8 with compound 9 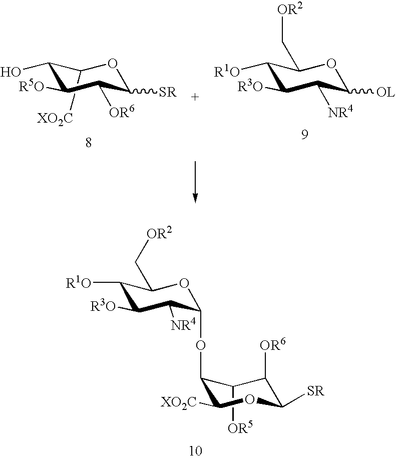
wherein R is an optionally substituted aryl group or an optionally substituted saturated or unsaturated alkyl group; R¹ to R⁶ are each independently the same or different protecting groups; X is selected from the group consisting of hydrogen, alkyl and amino; and L is a leaving group.

110. A process according to claim 109, wherein the process comprises reacting compound 8′ with compound 9′ to provide disaccharide 10′

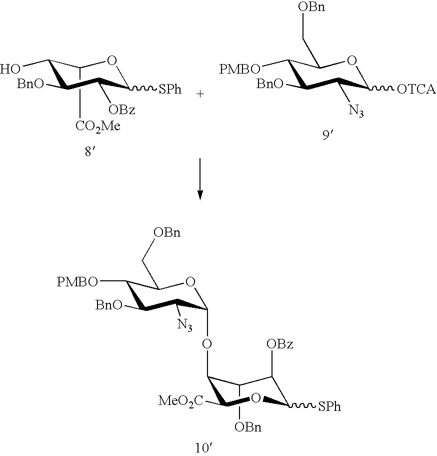

111. A disaccharide 10 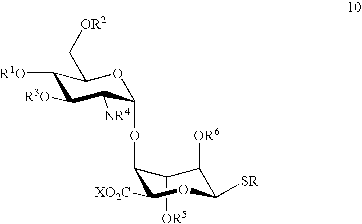
wherein R is an optionally substituted aryl group or an optionally substituted saturated or unsaturated alkyl group; R¹ to R⁶ are each independently the same or different protecting groups; and X is selected from the group consisting of hydrogen, alkyl and amino.

112. A disaccharide 10′

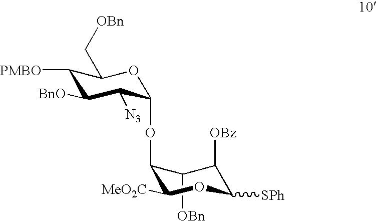

113. A process for the production of compound 8 from compound 7 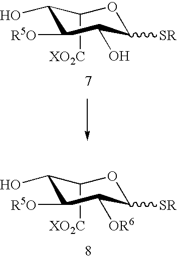
wherein R is an optionally substituted aryl group or an optionally substituted saturated or unsaturated alkyl group; R⁵ and R⁶ are each independently the same or different protecting groups; and X is selected from the group consisting of hydrogen, alkyl and amino; said process comprising adding a protecting group R⁶ to the 2-O atom of compound 7.

114. A compound 8 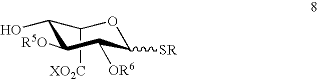
wherein R is an optionally substituted aryl group or an optionally substituted saturated or unsaturated alkyl group; R⁵ and R⁶ are each independently the same or different protecting groups; and X is selected from the group consisting of hydrogen, alkyl and amino.

115. A compound 8′

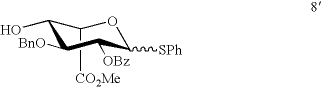

116. A process for the production of compound 7 from compound 6 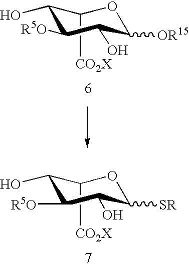
wherein R is an optionally substituted aryl group or an optionally substituted saturated or unsaturated alkyl group; R⁵ is a protecting group; R¹⁵ is an alkyl group; and X is selected from the group consisting of hydrogen, alkyl and amino.

117. A compound 7 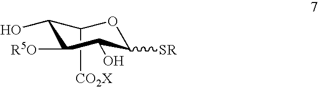
wherein R is an optionally substituted aryl group or an optionally substituted saturated or unsaturated alkyl group; R⁵ is a protecting group; and X is selected from the group consisting of hydrogen, alkyl and amino.

118. A process for the production of compound 8 from compound 6 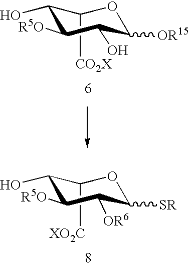
wherein R is an optionally substituted aryl group or an optionally substituted saturated or unsaturated alkyl group; R⁵ and R⁶ are each independently the same or different protecting groups; R¹⁵ is an alkyl group; and X is selected from the group consisting of hydrogen, alkyl and amino.

119. A process according to claim 118, wherein compound 8 has the formula 8′

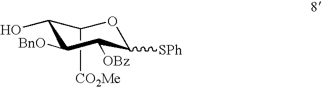

120. A process for the production of compound 9 from compound 11 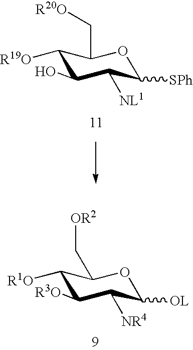
wherein R¹ to R⁴, R¹⁹ and R²⁰ are each independently the same or different protecting groups, R¹⁹ and R²⁰ may be linked; and L and L¹ are each independently the same or different leaving group.

121. A process according to claim 120, wherein compound 9 has formula 9′

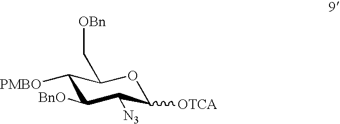

122. A process for the conversion of polysaccharide 20 to polysaccharide 26 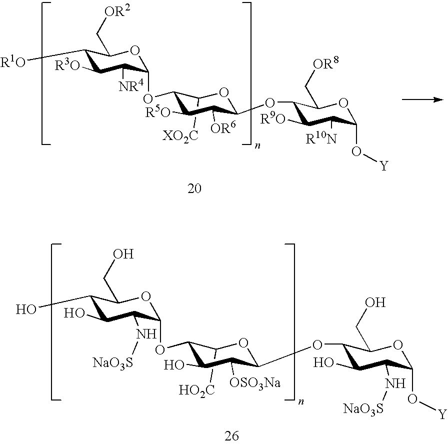
wherein R¹ to R⁶ and R⁸ to R¹⁰ are each independently the same or different protecting groups; X is selected from the group consisting of hydrogen, alkyl and amino; Y is selected from the group consisting of a protecting group and one or more saccharide residues; and n is a positive integer.

123. A process according to claim 122, wherein the process involves the conversion of polysaccharide 15 to polysaccharide 17 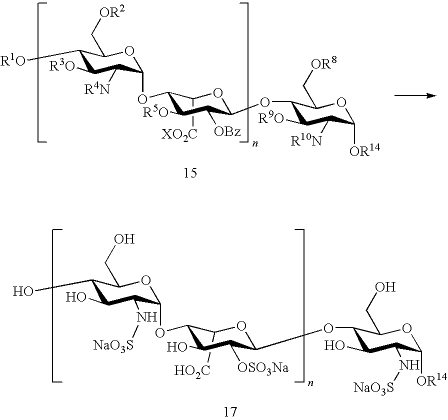
wherein R¹ to R⁵, R⁸ to R¹⁰ are each independently the same or different protecting groups; R¹⁴ is a substituted or unsubstituted aryl group or a substituted or unsubstituted saturated or unsaturated alkyl group; X is selected from the group consisting of hydrogen, alkyl and amino; and n is a positive integer.

124. A process according to claim 122, wherein the process involves the conversion of compound 16 to compound 18 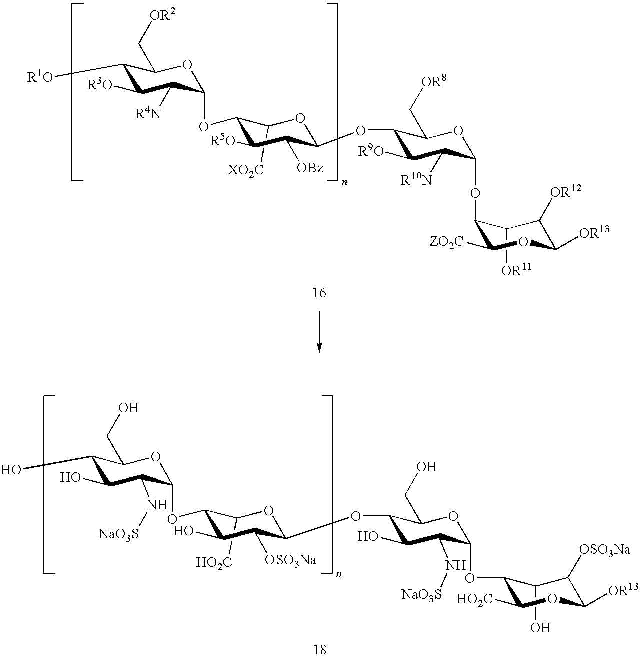
wherein R¹ to R⁵, R⁸ to R¹³ are each independently the same or different protecting groups; X and Z are each independently selected from the group consisting of hydrogen, alkyl and amino; and n is a positive integer.

125. A process for the conversion of compound 26 to compound 27 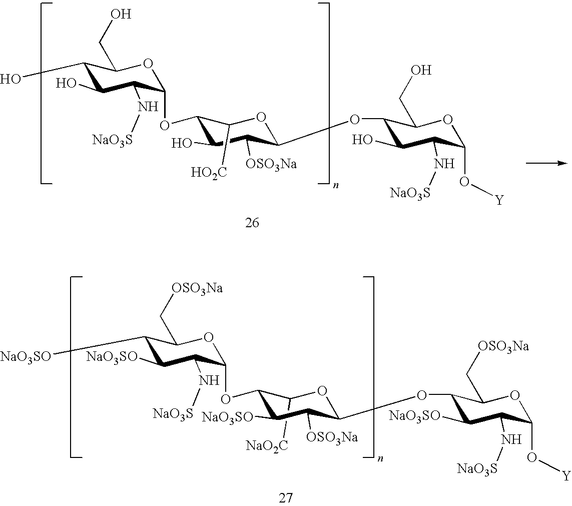
wherein Y is selected from the group consisting of a protecting group and one or more saccharide residues; and n is a positive integer.

126. A process according to claim 125, wherein the process involves the conversion of compound 17 to compound 19 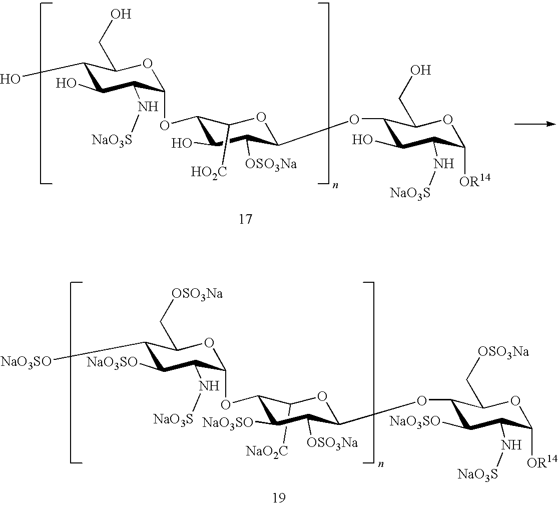
wherein R¹⁴ is a substituted or unsubstituted aryl group or a substituted or unsubstituted saturated or unsaturated alkyl group; and n is a positive integer.

127. A process according to claim 125, wherein the process involves the conversion of compound 18 to compound 23 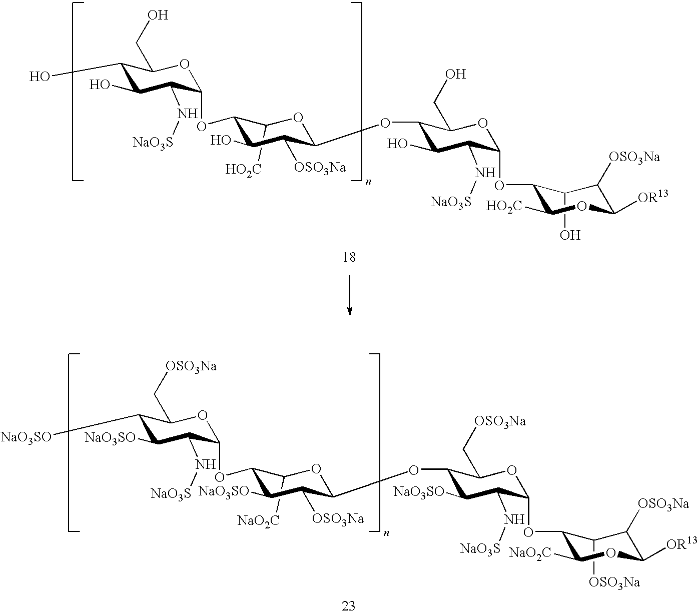
wherein R¹³ is a protecting group; and n is a positive integer.
